(19) United States
(12) Patent Application Publication
(10) Pub. No.: US 2024/0415560 A1
(43) Pub. Date: Dec. 19, 2024

(54) Vapor Ablation System with Simplified Control Over Vapor Delivery

(71) Applicant: Aqua Medical, Inc., Santa Ana, CA (US)

(72) Inventors: Scott McGill, San Ramon, CA (US); Lloyd Mencinger, Rancho Palos Verdes, CA (US); Virender K. Sharma, Paradise Valley, AZ (US)

(21) Appl. No.: 18/640,360

(22) Filed: Apr. 19, 2024

Related U.S. Application Data
(63) Continuation-in-part of application No. 17/150,188, filed on Jan. 15, 2021.
(60) Provisional application No. 63/497,678, filed on Apr. 21, 2023.
(60) Provisional application No. 62/961,473, filed on Jan. 15, 2020.

(51) Int. Cl. A61B 18/04; A61B 18/00

(52) U.S. Cl. CPC A61B 18/04; A61B 2018/00577; A61B 2018/00708; A61B 2018/0072; A61B 2018/00761; A61B 2018/00767; A61B 2018/00779; A61B 2018/00827; A61B 2018/00875; A61B 2018/00892; A61B 2018/048

(57) ABSTRACT

Ablation systems and methods include an improved approach to generating heated vapor. The system includes a controller having a user interface and receives data indicative of a treatment time or desired energy level to be delivered during a treatment session, a pump in data communication with the controller, and a catheter having a bipolar electrode in fluid communication with the pump. The controller is configured to control a delivery of fluid and a generation of heated vapor based on the data indicative of the treatment time or desired level of energy to be delivered without modifying the flow rate of the fluid or the level of voltage and/or current of the electrical current based on data from sensors positioned in or on the catheter.

### CROSS-REFERENCE

The present application relies on U.S. Patent Provisional Application No. 63/497,678, titled “Vapor Ablation System with Simplified Control Over Vapor Delivery” and filed on Apr. 21, 2023, for priority.

The present application is also a continuation in part application of U.S. patent application Ser. No. 17/150,188, titled “Vapor Ablation System with Simplified Control Over Vapor Delivery” and filed on Jan. 15, 2021, which relies on U.S. Patent Provisional Application No. 62/961,473, titled “Vapor Ablation System with Improved Control Over Vapor Quality and Delivery” and filed on Jan. 15, 2020, for priority.

The aforementioned applications are all hereby incorporated by reference in their entirety.

### FIELD

The present specification relates to systems and methods configured to simplify the generation and delivery of vapor for ablation-based therapy. More particularly, the present specification relates to systems and methods for creating and delivering a continuous and reliable stream of ablative vapor for focused and consistent tissue ablation using either treatment time or a total energy limit as a singular data input driving all subsequent operational variables to achieve the treatment.

### BACKGROUND

Ablation, as it pertains to the present specification, relates to the removal or destruction or modulation, (e.g. shrinking, tightening, remodeling, denaturing etc.), of a body tissue, via the introduction of a destructive agent, such as radiofrequency energy, laser energy, ultrasonic energy, cyroagents, or vapor, such as steam. Ablation is commonly used to reduce, compact, convert or eliminate diseased or unwanted tissues, such as, but not limited to cysts, polyps, tumors, hemorrhoids, and other similar lesions.

Steam-based ablation systems, such as the ones disclosed in U.S. Pat. Nos. 9,615,875, 9,433,457, 9,376,497, 9,561,068, 9,561,067, and 9,561,066, which are incorporated herein by reference, disclose ablation systems that controllably deliver steam through one or more lumens toward a tissue target. One problem that all such steam-based ablation systems have is the complex interaction of multiple variables which, if not properly managed, result in the inconsistent or unreliable delivery of vapor through catheter ports, and/or the potential overheating or burning of healthy tissue. Most steam-based ablation systems use resistive heating, which requires first delivering energy to a wire or metallic surface, heating the wire or surface, and, via the hot wire, vaporizing the ablation fluid. Resistive heating methods require waiting for the wire or surface to cool down in order to stop vaporizing fluid. These systems and methods often result in inconsistent and/or unreliable delivery of ablation vapor due to variability in parameters such as the heat transfer or surface area.

Furthermore, the effective use of steam often requires controllably exposing a volume of tissue to steam. However, prior art approaches to steam ablation either fail to sufficiently cover a volume being treated, thereby insufficiently exposing the tissue, or excessively enclose a volume being treated, thereby dangerously increasing pressure and/or temperature within the patient's organ. Pressure sensors located on the catheter may help regulate energy delivery, but they are not necessarily reliable and represent a critical point of potential failure in the system.

Additionally, prior art systems are often excessively costly or complex because they require the use of one or more sensors in a disposable catheter to monitor a vapor quality or temperature, and/or a temperature or a pressure of a body tissue or area being ablated. The inclusion of such sensors increases the cost and complexity of the ablation system and requires a user to monitor for any changes in temperature and/or pressure.

It is therefore desirable to have steam-based ablation devices that integrate into the device itself safety and/or vapor control mechanisms that are simple, result in the reliable delivery of steam, and prevent unwanted burning during use. It is further desirable to be able to provide a way to better control the amount of steam to which a target tissue is exposed without relying on sensors positioned within a catheter. It is also desirable to be able to provide an automated control of steam quality without requiring feedback from sensors. Such a system could avoid the use of sensors such as those for sensing pressure, temperature, vapor quality, moisture, or any other parameter for ensuring appropriate delivery of heat.

In case conventional steam-based ablation systems encounter a technical failure and stop operating, the vapor stored in the catheter is likely to burn the patient. Therefore, it is also desirable to have a heat delivery system that delivers the heat in a manner that avoids storage of a large amount of heat that could burn the patient.

Finally, current ablation systems have inflexible port structures that make it difficult to deliver vapor directly from a catheter to tissue that is positioned largely parallel to or skew to the port in a manner that avoids losing a substantial amount of vapor. Further, it is difficult to ablate using circular footprints, as there may be gaps of untreated areas left after ablating at adjacent locations, or there may be uncontrolled overlap in ablation, both situations being inefficient and possibly dangerous. Therefore, there is also a need to enable controlled and efficient ablation over defined surface areas that may be easy to minimize overlap and does not miss any of the target spaces under direct visualization. It is also desirable to provide steam-based ablation systems and methods used to treat various conditions including pre-cancerous or cancerous tissue in the esophagus, duodenum, bile duct, pancreas, or other tissues within the gastrointestinal system.

### SUMMARY

The present specification discloses a method of performing an ablation procedure, comprising: inputting at least one of a treatment time or an energy level into a controller, wherein the controller is in fluid communication with a pump and in electrical communication with a catheter and wherein the catheter has an elongate shaft, a proximal end, and a distal end, at least one lumen within the elongate shaft and at least one bipolar electrode positioned within the lumen; causing the controller to generate an electrical current and direct the electrical current to the at least one bipolar electrode such that the electrical current passes through the fluid positioned proximate the at least one bipolar electrode and causes the fluid to be transformed to a heated vapor, wherein the fluid is not fully transformed to heated vapor via resistive heating of the at least one bipolar electrode; and controlling a flow rate of the fluid and at least one of a level of power, voltage, current, and a time of treatment, based on data indicative of at least one of the time or the energy level to be achieved within the treatment session.

Optionally, the method further comprises not using temperature sensors or quality of steam sensors in the catheter.

Optionally, the method further comprises, prior to initiating treatment of a patient, using the controller to apply a known voltage and measuring a resulting current. Optionally, the method further comprises determining power as a function of the known voltage and the measured resulting current. Optionally, the method further comprises determining a preferred time for the ablation procedure based on a target amount of power and the determined power and performing the ablation procedure using the preferred time. Optionally, the method further comprises determining a preferred energy for the ablation procedure based on a target amount of power and the determined power and performing the ablation procedure using the preferred energy. Optionally, the controller is configured to terminate electrical current generation when a total energy level associated with all of the heated vapor has reached the preferred energy.

Optionally, the controller is configured to cause a first flow rate of the fluid before a generation of the heated vapor starts and a second flow rate of the fluid after the generation of the heated vapor starts, wherein the first flow rate is greater than the second flow rate. Optionally, the controller is configured to cause a third flow rate of the fluid after the preferred energy has been achieved, wherein the third flow rate is greater than the second flow rate.

Optionally, the method further comprises directing heated vapor through a cap in fluid communication with the distal end of the catheter, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater. Optionally, the cap comprises rounded exterior edges and is removably attachable to the distal end of the catheter. Optionally, the sole opening is polygonal in shape and the polygonal shape comprises one of a square, a rectangle, a pentagon, or a hexagon. Optionally, the cap is angled relative to the longitudinal axis of the catheter in a range of 5 degrees to 90 degrees.

Optionally, the catheter further comprises at least one first positioning element comprising a first disk spaced from a second positioning element comprising a second disk wherein the at least one bipolar electrode is positioned within the lumen between the first circular disk and the second circular disk, such that the heated vapor is generated in the space between the first circular disk and the second circular disk.

Optionally, the method further comprises detecting a start of heated vapor generation by monitoring a change in at least one of output power, output voltage, resistance, and output current and not based on an initiation of a flow of fluid.

Optionally, the fluid comprises sodium chloride in a range of 0.1% to 50% in water.

Optionally, delivering power to the at least one bipolar electrode in a range of 1 watts to 500 watts, wherein the power is based on at least one of a size of the lumen and a surface area of the at least one bipolar electrode.

Optionally, the catheter does not comprise sensors configured to detect data indicative of vapor quality, temperature, moisture level, or pressure of the heated vapor.

Optionally, the method further comprises causing the heated vapor to be generated for a first time period, ceasing a delivery of the fluid for a second time period, and repeating the generation of heated vapor and ceasing of fluid delivery for a plurality of cycles.

Optionally, the catheter comprises a programmable element and wherein the controller is configured to program the programmable element based on at least one of a treatment type, the power level, the voltage level, the current level, the fluid flow rate, the treatment time, or the energy level to be achieved. Optionally, the programmable element is a resistor.

The present specification also discloses a vapor ablation system comprising: a controller having a user input configured to receive data indicative of at least one of a treatment time or an energy level to be achieved within a treatment session; a pump in data communication with the controller; and a catheter in fluid communication with the pump and having an elongate shaft, a proximal end, and a distal end, the catheter comprising: at least one lumen; and at least one bipolar electrode within the lumen, wherein the controller is configured to control the pump to provide a fluid to the lumen of the catheter, wherein the fluid is a saline, and wherein the controller is configured to cause an electrical current to be delivered to at least one bipolar electrode in order to heat the fluid in the lumen and convert the fluid to a heated vapor, and wherein the controller is configured to control a delivery of the fluid and a generation of the heated vapor by controlling a flow rate of the fluid and a level of power, voltage and/or current, and a time of treatment, based on the data indicative of at least one of the time or the energy level to be achieved within the treatment session.

Optionally, the controller is configured to use the data to determine whether the energy level associated with the heated vapor has reached the energy level to be achieved within the treatment session.

Optionally, the controller is further configured to control the delivery of the fluid and the generation of the heated vapor by controlling at least one of the flow rate of the fluid and the level of power, voltage and/or current, and time of generation of the heated vapor.

Optionally, the controller is configured to increase the flow rate of the fluid during a period of time until the generation of the heated vapor starts.

Optionally, the controller is configured to increase the flow of fluid when the energy level to be achieved is achieved.

Optionally, the controller is configured to gradually decrease the level of power when the energy level to be achieved is achieved. Optionally, the controller is configured to increase the flow rate of the fluid during the gradual decreasing of the level of power when the energy level to be achieved is achieved.

Optionally, the vapor ablation system further comprises a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater. Optionally, the cap comprises rounded or curved exterior edges or surfaces and is removably attachable to the distal end of the catheter. Optionally, the sole opening has a footprint that is polygonal in shape. Optionally, the polygonal shape comprises one of a square, a rectangle, a pentagon, or a hexagon. Optionally, the side of the cap is angled relative to the longitudinal axis of the catheter in a range of 5 degrees to 90 degrees.

Optionally, the distal end of the catheter comprises a first circular disk spaced at a distance in a range from 1 to 5 centimeters from a second circular disk, such that the heated vapor is generated in a segment interspersed between the first circular disk and the second circular disk.

Optionally, the controller is further configured to detect an actual start of heated vapor generation, as independent and separate from an initiation of fluid flow to the at least one bipolar electrode, by monitoring a change in output power, output voltage, fluid resistance, or output current.

Optionally, the fluid comprises Sodium Chloride in a range of 0.1% to 50% in water.

Optionally, the controller is configured to deliver a power to the at least one bipolar electrode is in a range of 1 watts to 500 watts, wherein the power is based on at least one of a size of the lumen and a surface area of the at least one bipolar electrode.

Optionally, the controller is configured to deliver a flow rate of fluid into the lumen of 2.2 ml per minute.

Optionally, the controller is configured to deliver a voltage or current level associated with the rate of fluid delivery into the lumen to result in controlled steam vapor quality.

Optionally, the controller is configured to deliver a voltage or current level associated with the rate of fluid delivery into the lumen to result in high steam vapor quality.

Optionally, the controller is configured to deliver a voltage or current level associated with the rate of fluid delivery into the lumen to result in an prespecified average power level. Optionally, the controller is configured to deliver the fluid at at least one of a voltage and a current or a power level, and at an associated rate of fluid delivery at the average power level for a first time period, cease the delivery of the fluid for a second time period, and wherein the process of fluid delivery for the first time period and the second time period is repeated for a fixed number of cycles. Optionally, the controller is configured to increase the rate of fluid delivery during for a third time period, then deliver a voltage, a current or a power level and associated rate of fluid delivery at the prespecified average power level for a fourth time period, cease the delivery of the fluid for a fifth time period, and wherein the process of fluid delivery for the fourth time period and the fifth times period is repeated.

Optionally, the catheter does not comprise sensors configured to sense vapor quality, temperature, moisture level, or pressure of the heated vapor.

Optionally, the catheter comprises a programmable element wherein the controller is configured to program the programmable element based on at least one of a treatment type, power level, voltage level, current level, fluid flow rate, the treatment time, or the energy level to be achieved. Optionally, the programmable element is a resistor.

The present specification also discloses a vapor ablation system comprising: a controller having a user input configured to receive data indicative of a time of a treatment delivery or a total energy level to be achieved within the treatment session; a pump in data communication with the controller; and a catheter in fluid communication with the pump and having an elongate shaft, a proximal end, and a distal end, the catheter comprising: at least one lumen; and at least one electrode within the lumen, wherein the controller is configured to control the pump to provide a fluid to the lumen of the catheter, wherein the fluid is a saline, and wherein the controller is configured cause an electrical current to be delivered to at least one bipolar electrode in order to heat the fluid in the lumen and convert the fluid to a heated vapor, and wherein the controller is configured to control a delivery of the fluid and a generation of the heated vapor by controlling a flow rate of the fluid and a level of power, voltage and/or current based solely on the time of the treatment delivery or the total energy level to be achieved within the treatment session. Optionally, the controller is configured to collect data real-time during the treatment and adjust a treatment session end point based on the collected data.

Optionally, the controller is further configured to control the delivery of the fluid and the generation of the heated vapor by controlling the flow rate of the fluid and the level of power, voltage and/or current without modifying the flow rate of the fluid or the level of voltage and/or current based on data from sensors positioned in or on the catheter.

Optionally, the vapor ablation system further comprises a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater. Optionally, the cap comprises rounded or curved exterior edges or surfaces and is removably attachable to the distal end of the catheter. Optionally, the sole opening has a footprint that is polygonal in shape. Optionally, the polygonal shape comprises one of a square, a rectangle, a pentagon, or a hexagon. Optionally, the side of the cap is angled relative to the longitudinal axis of the catheter in a range of 5 degrees to 90 degrees.

Optionally, the controller is further configured to detect an actual start of heated vapor generation, as independent and separate from an initiation of fluid flow to the at least one electrode, by monitoring a change in output power, output voltage, or output current.

Optionally, the controller is further configured to detect an actual start of heated vapor generation, as independent and separate from an initiation of output power, output voltage, or output current to the at least one electrode, by monitoring a change in output power, output voltage, output current, and/or impedance of the conduit (i.e. saline).

Optionally, the controller is further configured to automatically apply a predefined on/off duty cycle for the time of a treatment session.

Optionally, the controller is further configured to automatically apply a predefined quantity of energy for the each treatment delivery.

Optionally the controller is further configured to allow the treatment time or total energy limit to be adjusted by the operator via an input to the controller.

Optionally, the controller is configured to adjust the syringe pump flow and the power output near the termination of the vapor delivery to minimize the strength of any vacuum phenomenon within the catheter lumen when vapor is terminated

Optionally, the controller is configured to deliver a power to the at least one electrode is in a range of 5 watts to 10,000 watts.

Optionally, the controller is configured to deliver a flow rate of fluid into the lumen of a range within 0.01 to 100 ml per minute.

Optionally, the controller is configured to automatically apply a fixed voltage or current/flow rate relationship during the treatment session that is not changeable based on sensed data indicative of a vapor quality, temperature, moisture level, or pressure of the heated vapor.

Optionally, the catheter does not comprise sensors configured to sense vapor quality, temperature, moisture level, or pressure of the heated vapor.

Optionally, the catheter comprises a programmable element and the controller is configured to program the programmable element based on at least one of a treatment type, power level, voltage level, current level, fluid flow rate, the treatment time, or total energy delivery limit. Optionally, the programmable element is a resistor, RF identification element, flash memory, or programable memory component.

Optionally, the catheter comprises a selectable element and the controller is configured to read the selectable element based on at least one of a treatment type, power level, voltage level, current level, fluid flow rate, treatment time, or total energy limit. Optionally, the selectable element is a resistor, RF identification element, flash memory, or programable memory component.

The present specification also discloses a vapor ablation system comprising: a controller having a user interface configured to receive data indicative of a time of a treatment session or a total delivered energy or a steam vapor delivered energy; a syringe pump in data communication with the controller; a catheter in fluid communication with the syringe pump and having an elongate shaft, a proximal end, and a distal end, the catheter comprising: at least one lumen; at least one electrode within the lumen, wherein the controller is configured to control the pump to provide a fluid to the lumen of the catheter, wherein the controller is configured to cause an electrical current to be delivered to at least one electrode in order to heat the fluid in the lumen and convert the fluid to a heated vapor, wherein the controller is configured to control a delivery of the fluid and a generation of the heated vapor by controlling a flow rate of the fluid and a level of power, voltage and/or current of the electrical current based on the data indicative of the time, and wherein the controller is further configured to control the delivery of the fluid and the generation of the heated vapor without modifying the flow rate of the fluid or the level of voltage and/or current of the electrical current based on data from sensors positioned in or on the catheter; and a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater.

Optionally, the cap includes a first circular disk spaced at a distance in a range from 1 to 5 centimeters from a second circular disk, such that the heated vapor is generated in a segment interspersed between the first circular disk and the second circular disk.

Optionally, the circular disk comprises a shape memory alloy structure and is configured to be mechanically deployed and retracted.

Optionally, the controller is configured to use the data to determine a duration for which the at least one electrode receive electrical current to heat the fluid in the lumen and convert the fluid to heated vapor.

Optionally, the vapor ablation system is adapted to operate at a fixed rate of flow of fluid from the pump to the lumen of the catheter.

Optionally, the controller is configured to maintain a voltage, current, or power level delivered to the at least one electrode at a steady state by maintaining a voltage level at a steady state.

Optionally, the controller is configured to maintain the rate of vapor production at a steady state by maintaining the rate of flow of the fluid and the salinity at a steady state.

The present specification also discloses a vapor ablation system comprising: a controller; a pump in data communication with the controller; a catheter in fluid communication with the pump and electrical communication with the controller; and having an elongate shaft, a proximal end, and a distal end, the catheter comprising: at least one lumen; at least one electrode within the lumen, wherein the pump is configured to provide a fluid to the lumen of the catheter, wherein the at least one electrode is configured to receive an electrical current from the controller to heat the fluid in the lumen and convert the fluid to a heated vapor, and wherein the controller is configured to control a quality of the heated vapor by controlling a level of voltage, current and/or power of the electrical control system based on a time input or energy limit and without relying on any temperature, pressure, moisture, or vapor quality sensors positioned on or within the catheter that is inserted into the patient's body; and, optionally, a cap in fluid communication with the distal end of the catheter and configured to direct ablative agent from the at least one lumen to a body tissue.

The fluid is a physiologically compatible fluid containing free ions, such as including but not limited to NaCl and Ca.

Optionally, the fluid is a sodium chloride and water solution, such as saline.

Optionally, the fluid is a physiologically normal saline.

Optionally, the controller is configured to deliver a power to the at least one electrode in a range of 1 watt to 10,000 watts.

Optionally, the controller is configured to deliver a flow rate of fluid into the lumen of the catheter in a range of 0.1-100 ml per minute.

Optionally, the at least one electrode comprises a bipolar electrode.

Optionally, the fluid is saline and the saline has a concentration of sodium chloride ranging from 0.01% to 50%.

Optionally, the cap comprises rounded or curved exterior edges or surfaces.

Optionally, the cap comprises an outlet footprint that is polygonal in shape. The polygonal shape may comprise one of a square, a rectangle, a pentagon, a hexagon, or other geometric shape.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1A illustrates an ablation system, in accordance with embodiments of the present specification;

FIG. 1B illustrates an ablation catheter in accordance with embodiments of the present specification;

FIG. 2A illustrates an ablation catheter for circumferential ablation in accordance with some embodiments of the present specification;

FIG. 2B illustrates a graph showing an inconsistent increase in temperature measured at vapor delivery ports of a circumferential ablation catheter;

FIG. 2C illustrates a graph showing a consistent increase in temperature measured at vapor delivery ports of a circumferential ablation catheter;

FIG. 3A illustrates an ablation catheter including a distal cap or focused ablation in accordance with embodiments of the present specification;

FIG. 3B illustrates an ablation catheter including a distal cap for focused ablation in accordance with other embodiments of the present specification;

FIG. 3C illustrates a cross-sectional side view distal cap attached to a distal end of an ablation catheter, in accordance with an embodiment of the present specification;

FIG. 3D illustrates a front-on view of a polygonal outlet of a distal cap, in accordance with an embodiment of the present specification;

FIG. 3E illustrates a front-on view of a polygonal outlet of a distal cap, in accordance with another embodiment of the present specification;

FIG. 3F illustrates a front-on view of a polygonal outlet of a distal cap attached to a distal end of an ablation catheter, in accordance with an embodiment of the present specification;

FIG. 3G illustrates a side view of a polygonal outlet of a distal cap attached to a distal end of an ablation catheter, in accordance with an embodiment of the present specification;

FIG. 4 is a flowchart listing the steps of a method of using an ablation system having a distal cap on an ablation catheter, in accordance with some embodiments of the present specification;

FIG. 5 is a flow chart illustrating an exemplary process of controlling generation of vapor in ablation device, in accordance with some embodiments of the present specification;

FIG. 6 shows an exemplary controller interface;

FIG. 7A shows an exemplary twisted pair or braided electrode, in accordance with some embodiments of the present specification;

FIG. 7B shows an exemplary twisted multi-wire or braided electrode, in accordance with some embodiments of the present specification;

FIG. 8 is a flow chart illustrating another exemplary process of controlling generation of vapor in ablation device, in accordance with some embodiments of the present specification; and

FIG. 9 is a flow chart illustrating another exemplary process of controlling generation of vapor in ablation device wherein the energy level is inputted to the controller, in accordance with some embodiments of the present specification.

### DETAILED DESCRIPTION

Embodiments of the present specification provide systems and methods of ablation therapy for treating a variety of conditions. The embodiments of the present specification describe ablation systems and methods that achieve a high degree of safety without having sensors embedded in the catheter for monitoring parameters such as temperature, moisture, pressure, and vapor quality and a minimized chance of burning or injuring a patient.

The embodiments of the present invention are intended to be deployed in known ablation systems. An exemplary known ablation system comprises a controller, having a pump (for example, a syringe pump) attached thereto, and a catheter, comprising an elongate shaft having a proximal end, a distal end, and at least one lumen within, attached via tubing to the controller and in fluid communication with the pump. The catheter and/or tubing are disposable and together form a disposable set. The catheter includes at least one electrode, positioned within a lumen of the catheter, to provide an energy source and convert a fluid (such as saline) into a vapor (such as steam) within the lumen. The at least one electrode is positioned at or proximate the distal end or tip of the catheter. The electrodes are positioned close to an output port on the catheter such that any vapor (steam) generated travels only a short distance (for example, a few centimeters) before exiting the catheter. The catheter is also in electrical communication with the controller for supply of power to the catheter in the form of an electrical current to the at least one electrode. The catheter includes a first electrical connection port, the controller includes a second electrical connection port, and at least one conductive wire connects the first electrical connection port to the second electrical connection port.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, microcontrollers, microprocessors, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the approaches to generating steam from water described in this application.

The terms “steam”, “water vapor”, “fluid vapor” and “vapor” are used interchangeably, and refer to the gaseous phase of a fluid that is used for ablation in accordance with the various embodiments of the present specification.

The term “steam quality” or “vapor quality” refers to a ratio of steam mass to liquid mass expressed as a percentage of total mass, of the vapor.

The term “flow rate” or “volumetric flow rate” is used interchangeably, and refers to the volume of fluid that passes through the catheter embodiments of the present specification.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

It should be appreciated that the devices and embodiments described herein are implemented in concert with a controller that comprises a microprocessor executing control instructions. The controller can be in the form of any computing device, including desktop, laptop, and mobile device, custom console and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

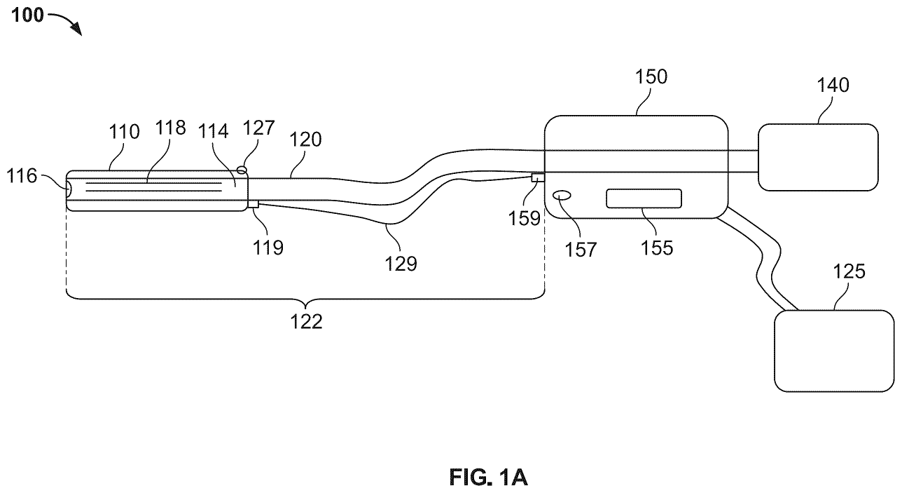

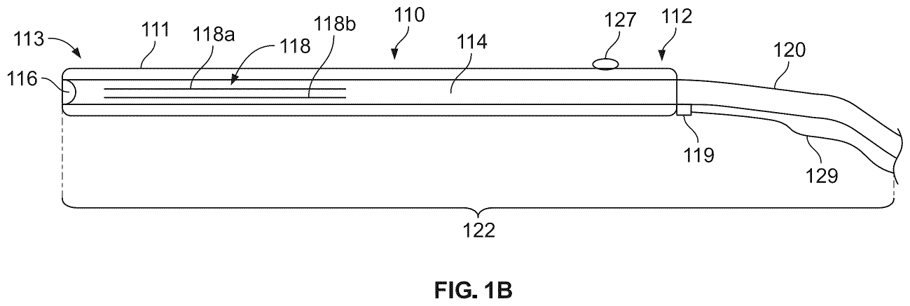

FIG. 1A illustrates an ablation system 100, in accordance with embodiments of the present specification. FIG. 1B illustrates a catheter 110 for use with the ablation system 100 of FIG. 1A. Referring to FIGS. 1A and 1B simultaneously, the ablation system 100 comprises a controller 150, a pump 140 attached to the controller 150, and a catheter 110, comprising an elongate shaft 111 having a proximal end 112, a distal end 113, and at least one lumen 114 within, attached via tubing 120 to the controller 150 and in fluid communication with the pump 140. The controller 150 includes a microprocessor 155 for controlling a rate of flow of ablative agent and the controller output. In embodiments, the system 100 comprises an input device 125 in data communication with the controller 150 configured to allow a user to adjust at least one or both of a treatment duration in number of seconds or minutes, and energy limit in Joules to be delivered. In some embodiments, the end or termination of a treatment duration is calculated and set by the system real-time as energy is delivered. In embodiments, the total energy is the average power multiplied by time. For example, in an embodiment, a catheter has an average power output (given the voltage and flow rate) of 50 Watts. The energy limit is set to 200 Joules. In this embodiment, the steam delivery will be terminated when the energy limit is reached in 4 seconds: 50 W×4 sec=200 J. In some embodiments, the input device comprises a foot pedal and/or a graphical user interface (GUI). In some embodiments, a switch 127 on the catheter 110, or a switch 157 on the controller 150, is provided and configured to allow a user to control the flow of ablative agent.

In one embodiment, the GUI is configured to allow a user to define device, organ, and condition which in turn creates default settings for key variables, such as temperature, cycling, volume, volumetric flow rate, power, time and standard energy or radiofrequency (RF) settings, for example-RF voltage, RF current, RF power, RF impedance. In one embodiment, these defaults can be further modified by the user. The user interface also includes standard displays of all key variables, along with warnings if values exceed or go below certain levels. In embodiments, the system 100 also includes safety mechanisms to prevent users from being burned while manipulating the catheter, including markings on the catheter shaft indicating the hot or steam generation zone, insulation, and optionally, cool air flush, cool water flush, and alarms/tones to indicate start and stop of treatment.

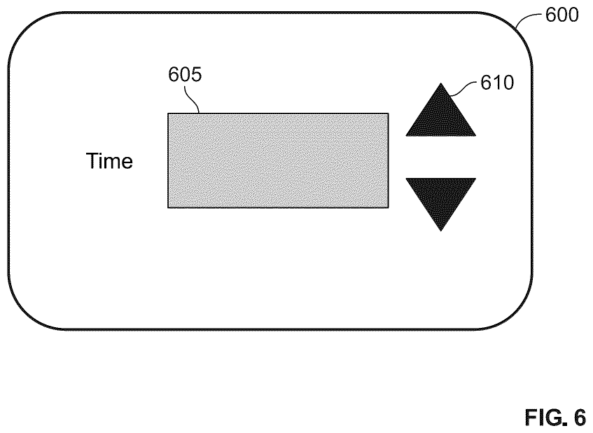

Referring to FIG. 6, in one embodiment, the controller comprises a graphical user interface 600 that just displays an input 610 configured to receive at least one or both of time and energy delivery setting 605 for a chosen treatment session, which is based on the tissue being ablated and/or the therapy being provided. In one embodiment, the inputting of time and/or energy setting, which may be determined based on the tissue being ablated and/or the therapy being provided, automatically and consequentially determines all further operational variables, including a flow rate, a voltage level, and/or a power level, as further described below.

In some embodiments, the pump 140 is a syringe pump. In various embodiments, heated vapor, generated by heating, within the catheter, an electrically conductive solution such as saline provided by the pump 140, is used as an ablative agent. Saline is preferred as a fluid for generating heated vapor, rather than water, as saline is a conductive fluid while water is not. A saline solution with a specific conductivity and resistivity is desired, as the solution requires resistivity so as to become hot and vaporize, and too high of a conductivity will prevent the heating. Additionally, surface area of the electrode is defined to optimize the amount of steam generated in a specific amount of time. Greater surface area of the electrode increases the amount of power that can be delivered, resulting in a greater amount of steam being generated in a similar amount of time with an electrode of a relatively smaller surface area. In embodiments, the electrode herein refers to a pair of bipolar electrodes, or two electrodes of opposite polarities, such that when current is passed through the electrode it travels from one bipolar electrode to the other bipolar electrode through the medium (typically conductive fluid such as saline) thereby energizing the medium. The temperature of the medium increases when the current is passed through the electrode. In embodiments, the rate of temperature increase is rapid when the current is in the form of radiofrequency alternating current (for example, 460-1000 kHz). In embodiments, saline with a sodium chloride concentration in a range of 0.01% to 50% is used to optimize conductivity. In one embodiment, saline with a sodium chloride (NaCl) concentration of 0.9% is used.

The catheter 110 includes at least one electrode (and preferably a pair of electrodes) 118, positioned within the lumen 114 of the catheter 110, to deliver energy for generating heat and convert a fluid into a vapor within the lumen 114. In some embodiments, catheter 110 includes at least two electrodes 118a and 118b of two opposing polarities. Saline flows between the two electrodes 118a and 118b, which is energized by the current delivered to one electrode that passes through the fluid to get to the second electrode of opposite polarity. Therefore, the fluid (saline) is vaporized by directly driving of energy into the fluid between the two electrodes 118a and 118b. The energy in the form of electric current (i) delivered by at least one electrode 118 heats the fluid by the current flowing through the resistive fluid (R) causing internal fluid heating, which converts some quantity of the fluid to vapor. In embodiments, the heat generated may be represented by the following power equation:

i²*R
where i=electric current (in Amps) and R=fluid resistance (in Ohms), with the resulting power unit in Watts.

The at least one electrode 118 is positioned at or proximate the distal end 113 or distal tip of the catheter 110. The at least one electrode 118 is positioned close to at least one output port 116 on the catheter 110 such that any vapor generated travels only a short distance before exiting the catheter 110. The catheter 110 is also in electrical communication with the controller 150 for supply of power to the catheter 110 in the form of an electrical current to the at least one electrode 118. In embodiments, the catheter 110 includes a first electrical connection port 119, the controller 150 includes a second electrical connection port 159, and a metal wire 129 connects the first electrical connection port 119 to the second electrical connection port 159. In some embodiments, the at least one electrode 118 comprises at least one pair of electrodes 118a, 118b or comprises at least one elongated bipolar electrode. In some embodiments, there are multiple independently controlled channels of bi-polar pairs of electrodes. In embodiments, the electrodes of the least one pair of electrodes 118a, 118b are cylindrical in shape and spaced apart from one another. Electrical current provided to the at least one electrode 118 results in a generation of heat to heat a fluid, such as saline, flowing in a lumen 114 and to convert the fluid into heated vapor. In some embodiments, multiple electrodes are positioned in “series” along the length of the catheter lumen 114. In some embodiments, multiple electrodes are positioned concentrically. In one embodiment, and as shown in FIGS. 1A and 1B, the electrode 118 has conductors on the top and bottoms sides, and fluid passes over both sides of the flat electrode assembly.

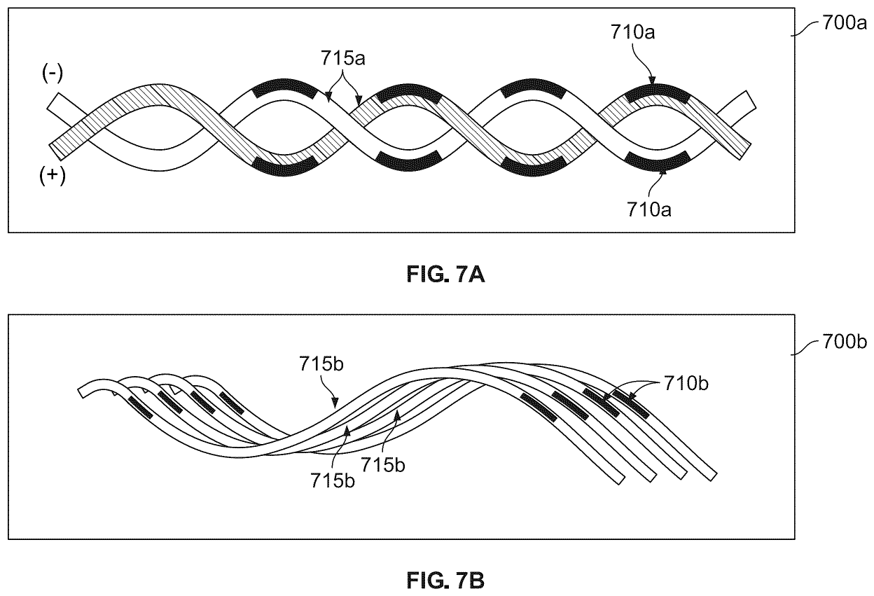

In one embodiment, referring to FIGS. 7A and 7B, the electrode 700a, 700b respectively, comprises a single twisted pair or a braid (comprising two helically twisted or interwoven wire segments 715a) or multi-twisted wires or multi-braid (comprising more than two helically twisted or interwoven wire segments 715b, preferably 3, 4, 5, 6 or more interwoven wire segments). Each wire segment in the twisted pairs or braids 715a, 715b comprises a conductive material that is covered by an insulating material, yielding twisted pairs or braids 715a, 715b having helically interwoven insulated individual wire segments. In selected locations along the length of the individual wire segments, the insulation is removed, exposing the underlying conductive material 710a, 710b. The exposed portions 710a, 710b of the individual wire segments of the twisted pairs or braids 715a, 715b are aligned such that, when evaluated along the longitudinal length of the electrode, the exposed portions of each individual wire segment may not overlap or may overlap in a range of 1% to 100%, or any increment therein.

In some embodiments, electrode configurations are defined by their respective surface areas and/or peripheral edges. In some embodiments, a vaporizing electric field is created by using a range of 1 to 25 bipolar pairs of electrodes, thereby providing a range of 4 to 100 edges, wherein four edges comprise a bipolar pair. In some embodiments, power to the at least one electrode is supplied at a range of 2 watts to 10,000 watts. In some embodiments, saline flow is supplied to the catheter at a range of 0.1 to 100 ml/min. In one embodiment, the ablation system operates at a 2.2 ml/min saline flow rate and a 50 watt average power level.

The ablation systems of the present specification operate using a power/flow rate relationship to control vapor quantity and/or steam quality without further relying on sensed data from the catheter, particularly temperature, pressure, moisture, steam quality or vapor quality data. More preferably, the power/flow rate and time is automatically triggered or set based on the desired ablative effects input in terms of treatment duration or energy level to be achieved within the treatment, as previously described above. Specifically, by having the at least one electrode 118 positioned proximate the at least one output port 116 on the catheter 110, steam quantity is tightly controlled as the steam is outputted almost immediately after being produced and steam quality can be controlled. Additionally, as a result of the electrode 118 positioned proximate the output port 116, a shorter segment of the catheter, proximate the port 116 is heated. Moreover, less heat loss results in better steam quality. A high steam quality is defined as a steam wherein the amount of vapor is equal to or higher relative to the amount of condensed water. In one embodiment, the heated vapor has a quality level of at least 0.10, preferably higher than 0.50, measured as the proportion of saturated vapor in a saturated condensate mixture. Steam quality can be maintained at a high level by controlling the amount of power, in the form of electrical current, being supplied to the at least one electrode 118, and by controlling a flow rate of fluid from the pump 140 to the catheter 110. As uniquely determined by the inventors, following a specific relationship between flow rate and power supplied ensures the generation of high-quality steam without requiring further control or modification by data inputted or received from one or more sensors positioned on or within the catheter. This avoids the need for any sensors positioned within, embedded within, or positioned along the length of the catheter, such as temperature, pressure, moisture, or fluid flow rate sensors, to monitor the steam quality and ensure the quality is sufficient for ablation and not out of range, possibly causing injury or under treatment. Steam quality is controlled by fine tuning two variables: a range of voltage or electrical current supplied to the at least one electrode and a range of saline flow rates. For example, a voltage setting of 35 volts and a flow rate of 2.2 ml/min may result in a vapor quality on the order of 40%-80% with a given catheter and electrode design or configuration. In a most preferred embodiment, the range of voltage supplied is 25 Volts (V) to 80V, the range of current supplied is 1 Ampere (A) to 5 A, the range of power supplied is 45 Watts (W) to 85 W, and the range of flow rate supplied is 1.8 milliliter per minute (ml/min) to 2.5 ml/min. These parameters result in a desired vapor ablation on a defined tissue surface. The total amount of energy, the vapor quality, and temperature and the time of vapor to tissue exposure result in the desired ablation effect. In a preferred embodiment, the range of voltage supplied (solely based on a desired time tissue effect input) is 10 Volts (V) to 55V, the range of current supplied (solely based on a desired tissue effect time input) is 0.5 Ampere (A) to 10 A, the range of power supplied (solely based on a desired tissue effect time input) is 20 Watts (W) to 100 W, and the range of flow rate supplied (solely based on a desired tissue effect time input) is 0.9 milliliter per minute (ml/min) to 4.0 ml/min.

The almost immediate generation and delivery of heated vapor from when it is produced, referred to as “just in time vapor generation and delivery”, ensures that there is a continuous flow of steam that is quickly delivered, resulting in very little heat stored within the catheter or controller and, accordingly, a safer system. In various embodiments, the ablation system stores heat at a value less than 500 J, preferably less than 100 J, defined by an amount of vapor or water equal to or less than a heated volume of 0.5 ml of saline, preferably less than 0.1 ml of saline. The embodiments of the present specification therefore provide a safety mechanism in case the system stops operating or fails, since there is no vapor that remains to be discarded. Therefore, the various embodiments prevent or dramatically reduce the risk of damage due to burns from stored heat.

In some embodiments, a patient is treated in a two-step process to ensure complete or near complete ablation of a target tissue. In some embodiments, a patient is first treated with a catheter having two positioning elements—a distal positioning element that is initially deployed followed by a proximal positioning element deployed thereafter, and a tube length with ports positioned between the two positioning elements, thereby enabling wide area circumferential ablation. In some embodiments, the two positioning elements are circular disks positioned at a linear distance in a range of 1 to 5 centimeters (cm) from each other. The ports are interspersed between the two circular disks, where the ports are outlets for delivery of ablation vapor. In some embodiments, the two positioning elements are configured to apply a radial force to the tissue of a tubular shaped organ.

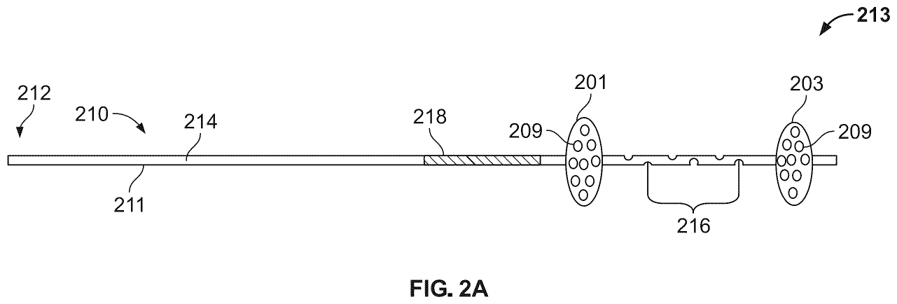

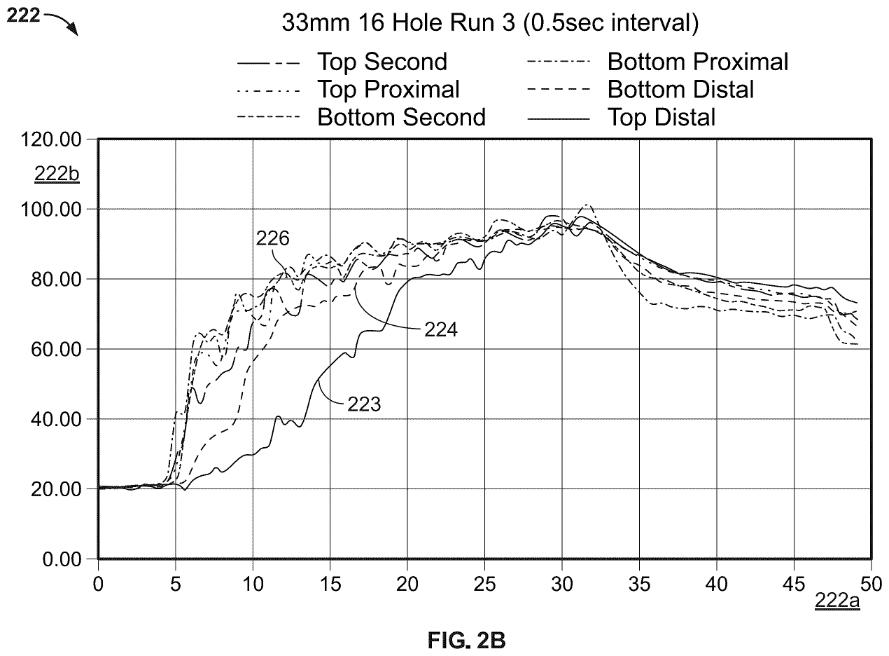

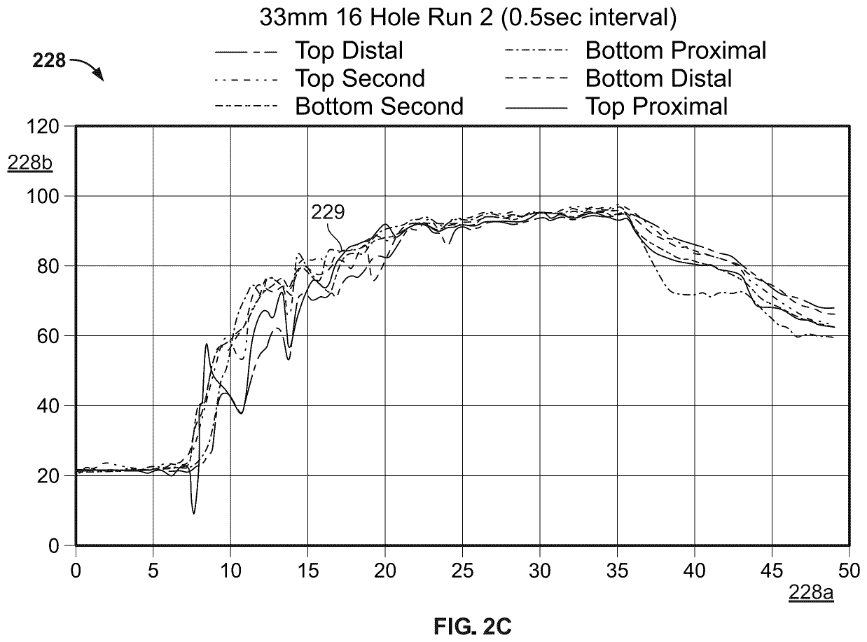

FIG. 2A illustrates an ablation catheter 200 for circumferential ablation in accordance with some embodiments of the present specification. The ablation catheter 210 includes an elongate shaft 211, a proximal end 212, a distal end 213, and at least one lumen 214. A proximal positioning element 201 is positioned proximate the distal end 213 and a distal positioning element 203 is positioned distal to the proximal positioning element 201. In some embodiments, the two positioning elements 201, 203 are circular disks positioned at a linear distance in a range of 1 to 5 centimeters (cm) from each other. The ports are interspersed between the two circular disks, where the ports are outlets for delivery of ablation vapor. A plurality of ports 216 are positioned on the catheter shaft 211 in between the proximal positioning element 201 and the distal positioning element 203. At least one electrode (preferably at least one pair of electrodes of opposite polarities) 218 is positioned in at least one lumen 214 for converting fluid to vapor. A first contact (but preferably no seal or meaningful blocking of vapor) is created by contact of the periphery of the positioning elements 201, 203 with a patient's tissue at said distal and proximal positioning elements 201, 203. Less preferably, a seal may be created. Ablative energy, in the form of steam, is then delivered by the catheter 210 via the ports 216 into the first treatment volume, where it contacts the patient's tissue and condenses for circumferential ablation and cannot escape from the distal or proximal ends as it is blocked by the positioning elements 201, 203 (less preferably) or, preferably, escapes from the distal or proximal ends based on the configuration of the positioning elements 201, 203 or the presence of small holes or channels 209 in the positioning elements 201, 203.

The ports 216, which extend between the two positioning elements, are configured such that a surrounding chamber receives a similar distribution of vapor. In embodiments, the size, shape, direction/angle and location of the ports can vary based on position to help optimize the distribution of vapor. For example, the rate of temperature increase measured at various points on the internal wall of a patient's gastrointestinal (GI) tract would be substantially equal across all points. This would prevent some surfaces from receiving too much thermal energy and other surfaces from receiving too little, ensuring generally uniform ablation.

FIG. 2B illustrates a graph 222 showing an inconsistent increase in temperature measured at vapor delivery ports of a conventional ablation catheter. An x-axis 222a illustrates the time, and a y-axis 222b illustrates the temperature (in ° C.). There are some points, depicted by curves 223, 224, that lag in terms of temperature increase relative to other points, depicted by a generally similar distribution of curves 226. In practice, these points will not get enough energy. FIG. 2C illustrates a graph 228 showing a relatively more consistent increase in temperature measured at vapor delivery ports of a circumferential ablation catheter in accordance with embodiments of the present specification. An x-axis 228a illustrates the time, and a y-axis 228b illustrates the temperature (in ° C.). The points, depicted by generally similar distribution of curves 229, shows a relatively consistent rate of increase across all points, meaning more consistent energy deposition across all surfaces.

In embodiments, the circumferential ablation catheters 210 of the present specification are configured to establish an array of points defined by a specific distance from a portion of the catheter shaft 211 such that each point will experience an increase in temperature at approximately the same rate. In other embodiments, the circumferential ablation catheters 210 of the present specification are configured to establish an array of points defined by a specific distance from a portion of the catheter shaft 211 such that each point on the tissue to be ablated will experience similar temperatures, from 60° C. to 90° C. to same depth, from 0.5 mm to 5 mm within five seconds of each other.

In embodiments, the ports 216 of the circumferential ablation catheters 210 of the present specification are configured such that a ratio of a surface area of port 216 openings to a surface area of catheter 210 length between the two positioning elements 201, 203 is less than 0.25, and preferably less than 0.10. The catheters 210 are configured to have a large number of holes, from 16 to 100, but not exceeding a percentage of surface area of the catheter shaft 211. In embodiments, each port 216 has a diameter ranging from 0.05 mm to 2 mm. In embodiments, the number of holes is inversely proportional to the size of each hole, to ensure a sufficient flow of vapor through the holes. Accordingly, as the port diameters increase in size from 0.05 mm to 2 mm, the total number of ports decrease. In embodiments, the hole spacing and the diameter of the holes may vary to optimize uniform vapor delivery within the defined treatment area.

In some embodiments, the circumferential catheters 210 include one or more features to avoid pooling of water in the patient's organ (GI tract). “Pooling” occurs when hot water (not just steam) drips out of the ports and gathers in areas of tissue which may not be subject to ablation. As the circumferential ablation catheter is substantially horizontal when in use since the patient is lying on his or her back, hot water pools may form below the catheter and in the dependent or bottom surfaces of the GI tract. Configurations of the catheters 210 provide a check on formation of pools. In one embodiment, an outer surface of the catheter is in electrical communication with the second electrical connection port (159 in FIG. 1A) of the controller to create a heated surface. The configuration of two electrical ports may ensure that the vapor coming out of ports 216 remain in a vapor state. Additionally, in some embodiments, the ports 216 are narrow slits instead of circular ports. Narrow slits are created using laser cutting. In some embodiments, slits improve flexibility of the length between positioning elements 201 and 203. In another embodiment, the ports 216 are concentrated in certain locations where pooling is expected to occur. For example, in one embodiment, more ports 216 are positioned toward the distal end 213 of the catheter. In less preferred embodiments, steam at the distal end 213 of the catheter is pressurized by decreasing the catheter lumen 214 size or superheating the steam when it exits the catheter 210 using trumpet like nozzles at the ports 206. In still another embodiment, the ports are covered with a semi-permeable or hydrophobic material that allows gas to pass but not liquid. In some embodiments, the material is polytetrafluoroethylene (PTFE). In various embodiments, any one or combination of the above mechanisms is used to avoid all forms of pooling. In some embodiments, the saline delivery tubing and the entire fluid pathway is constructed of non-expanding (i.e., pressure rated) materials to ensure the pathway is completely void of air. The absence of air in the system or the tubing helps prevent the expansion of the system/tubing under pressure during the delivery of steam. This in turn prevents, after the delivery of steam is stopped, the drippage and pooling of fluid out of the catheter when the expanded tubing recovers.

After circumferential ablation is performed in the first step, the ablation area is examined by the physician. Upon observing the patient, the physician may identify patches of tissue requiring focused ablation. In embodiments, a circular or polygonal ablation footprint can be created, but a polygonal ablation footprint is used to make the focused ablation more efficient relative to a circular ablation footprint. A circular footprint may result in the creation of gaps or overlaps while ablating adjacent areas, which may be inefficient. In embodiments of the present specification, the polygonal ablation footprint is used, which is easy to stack and unlikely to leave gaps. After examination of the circumferential ablation area by the physician, second step is performed to provide focused ablation. During focused ablation, a second catheter with a needle or cap, hood, or disc attachment on the distal end is passed through an endoscope and used for focal ablation. In embodiments, the cap has a round or a polygonal outlet surface area. The polygonal outlet surface area may be a square, a pentagon, a hexagon, or any other type of polygon.

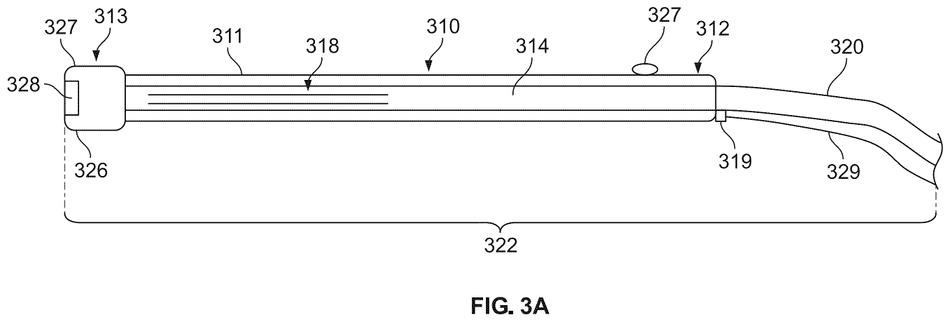

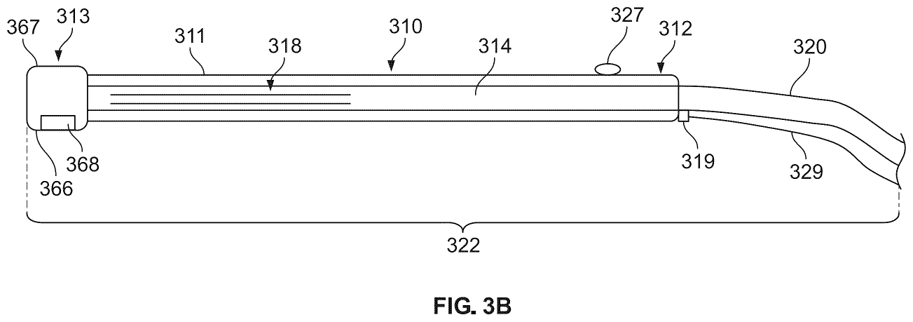

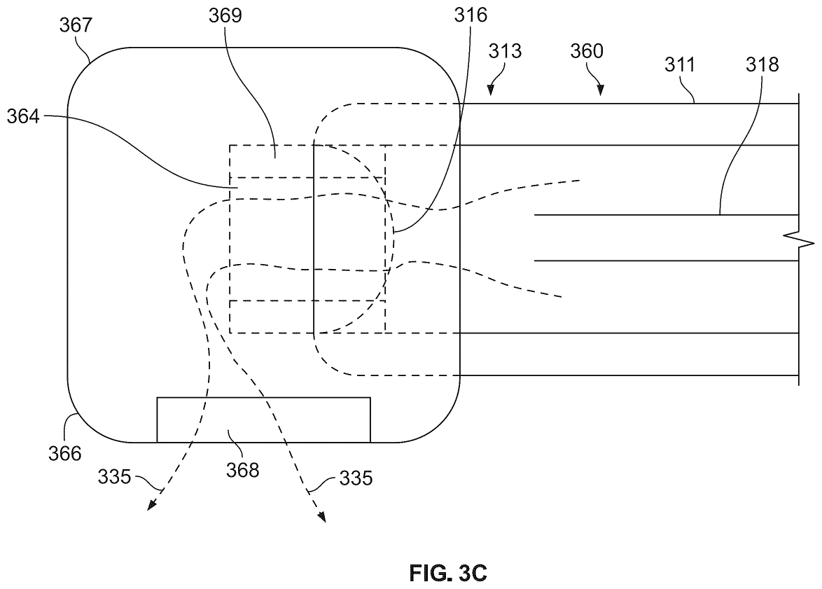

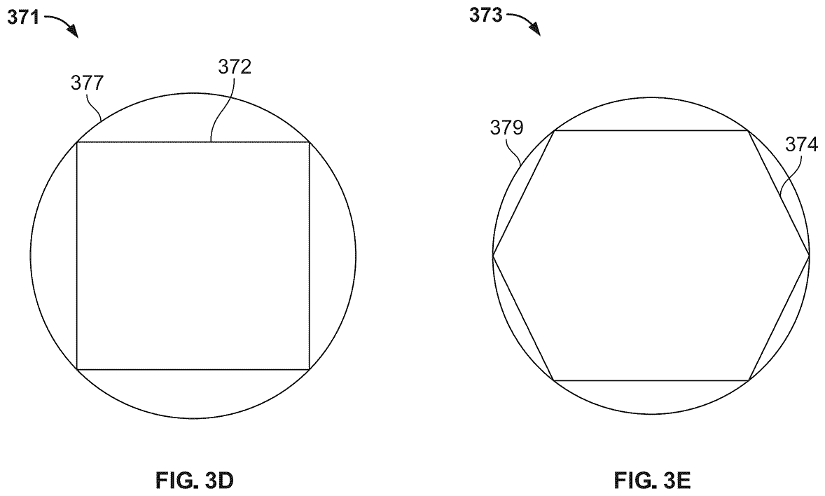

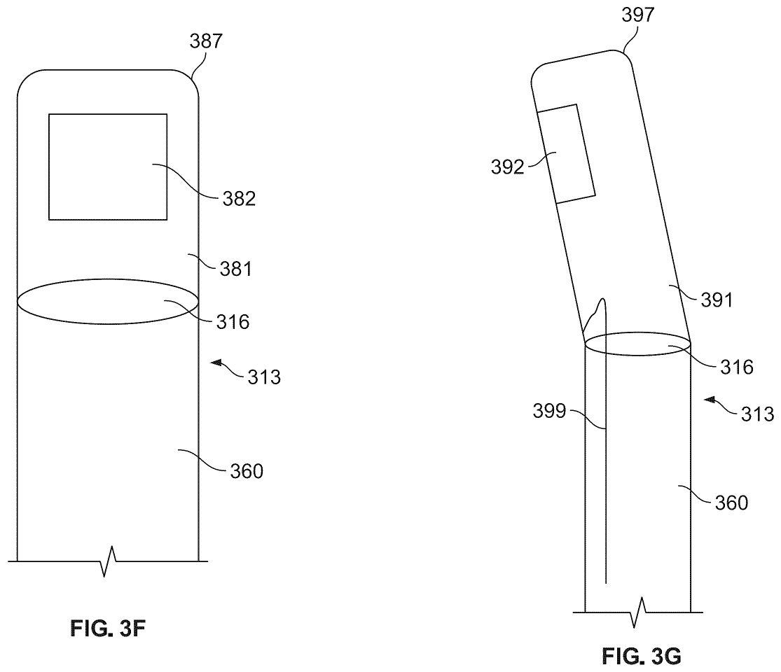

FIGS. 3A and 3B illustrate ablation catheters 310, 360 including a distal cap 326, 366 for focused ablation in accordance with embodiments of the present specification. In some embodiments, the cap 326, 366 is made of a collapsible, expanding material that can be inserted through an endoscope. In some embodiments, the cap 326, 366 can be a separate component attached to the endoscope or other surgical tools. Similar to the catheter 110 of FIG. 1B, the ablation catheters 310, 360 of FIGS. 3A and 3B include an elongate shaft 311 with a proximal end 312 and a distal end 313, at least one lumen 314 with at least one electrode 318 within, and a switch 327 for controlling vapor flow. The catheters 310, 360 are in fluid communication with a pump via tubing 320 and are in electrical communication with a controller via wire 329 connected to electrical connection port 319 at the proximal end 312. In some embodiments, the catheter 310, 360 and tubing 320 together form a disposable set 322. A distal cap 326, 366 is attached to the distal end 313 of the catheter 310, 360. The distal cap 326, 366 includes a round or a polygonal shaped outlet port 328, 368 for focused delivery of steam. In the embodiments depicted in FIGS. 3A and 3B, the outlet ports 328, 368 are rectangular or square shaped.

Referring to FIG. 3A, the outlet 328 is at a distal end of the distal cap 328. Referring to FIG. 3B, the outlet 368 is on a side of distal cap 366. It should be appreciated that the cap comprises a housing that fully encloses a volume, except for a window, which is a void or opening in the housing, positioned on a side of the cap such that it is parallel to the longitudinal axis of the catheter. An outer edge or surface 327, 367 of the distal cap 326, 366 is rounded or curved to provide an atraumatic tip and prevent injury, avoiding edges that are too sharp and could cut the patient's anatomy, for example, the gastrointestinal (GI) tract. In embodiments, the distal cap 326, 366 is enclosed except for the outlet 328, 368 that defines the ablation footprint which captures and concentrates the vapor. The footprint of outlet 328, 368 is shaped in the form of a circle or a polygon to allow for easy stacking without overlap.

FIG. 3C illustrates a cross-sectional side view of distal cap 366 attached to a distal end 313 of an ablation catheter 360 of FIG. 3B, in accordance with an embodiment of the present specification. A portion of the distal cap 366 slides over and covers a distal portion of the catheter shaft 311. The distal cap 366 includes a connector 369 with a lumen 364 that is configured to be inserted into an outlet port 316 of the catheter. The distal cap 366 includes a side outlet port 368 in a circular or a polygonal shape. Steam 335 is directed from the lumen 314 of the catheter 360, through outlet 316 and the lumen 364 of the connector 369, and out the side outlet port 368 for focused ablation. Position of the at least one electrode 318 proximate the distal end 313 of the catheter 360 ensures steam has a very short distance to travel to reach a target tissue after being generated. An outer edge or surface 367 of the distal cap 366 is rounded or curved to provide an atraumatic tip and prevent injury.

FIG. 3D illustrates a front view of a polygonal outlet 372 of a distal cap 371, in accordance with an embodiment of the present specification. The polygonal outlet 372 is square shaped and the distal cap 371 includes rounded or curved outer edges or surface 377 to provide an atraumatic tip and prevent injury. FIG. 3E illustrates a front-on view of a polygonal outlet 374 of a distal cap 373, in accordance with another embodiment of the present specification. The polygonal outlet 374 is hexagon shaped and the distal cap 373 includes rounded or curved outer edges or surface 379 to provide an atraumatic tip and prevent injury.

FIG. 3F illustrates a front view of a polygonal outlet 382 of a distal cap 381 attached to a distal end 313 of an ablation catheter 360, in accordance with an embodiment of the present specification. The polygonal outlet 382 is square shaped and the distal cap 381 includes rounded or curved outer edges or surface 387 to provide an atraumatic tip and prevent injury. Steam flows from the lumen of the catheter 360 through an outlet port 316 of the catheter 360, through the distal cap 381, and out the circular or polygonal outlet 382. FIG. 3G illustrates a side view of a polygonal outlet 392 of a distal cap 391 attached to a distal end 313 of an ablation catheter 360, in accordance with an embodiment of the present specification. The polygonal outlet 392 is rectangular shaped and the distal cap 391 includes rounded or curved outer edges or surface 397 to provide an atraumatic tip and prevent injury. Steam flows from the lumen of the catheter 360 through an outlet port 316 of the catheter 360, through the distal cap 391, and out the circular or polygonal outlet 392.

Referring to FIG. 3G, the distal cap is tilted or biased to one side, such that it is angled at least 1 degrees, preferably at least 5 degrees but less than 90 degrees, more preferably at least 10 degrees, more preferably in a range of 5 to 45 degrees, relative to the longitudinal axis of the catheter, allowing for an even more focused ablation of a target tissue. In some embodiments, the catheter 360 includes a mechanism 399 for tilting the distal cap 391 at a greater or lesser angle and for modifying the direction of the tilt. The tilted distal cap 391 with polygonal outlet 392 provides for easier positioning of the catheter 360 as the physician does not have to figure out how to bend or move the outlet surface to hit the desired target surface (given that, when first inserted, the outlet points downward, parallel to the GI tract). The physician is only required to gently push the polygonal outlet 392 against the GI tract for proper positioning. In some embodiments, the polygonal outlet 392 has a surface area in a range of 0.5 cm² to 5 cm².

The distal caps illustrated in FIGS. 3A-3G are configured to connect to the catheter distal end or tip. In some embodiments, the distal cap includes a groove and/or O-ring that attaches or snaps into the distal tip of the catheter. In some embodiments, the distal cap further comprises an additional channel that directs the vapor from the catheter lumen into the cap and toward the distal cap outlet port. In some embodiments, the catheter lumen is positioned off-center of the catheter shaft, and the distal cap further comprises a connecting member configured to insert into the catheter lumen and direct the vapor to the outlet port of the distal cap. In some embodiments, the distal cap channel has a length in a predefined range and a maximum thickness in a predefined range to fit into, and stay within, the catheter lumen.

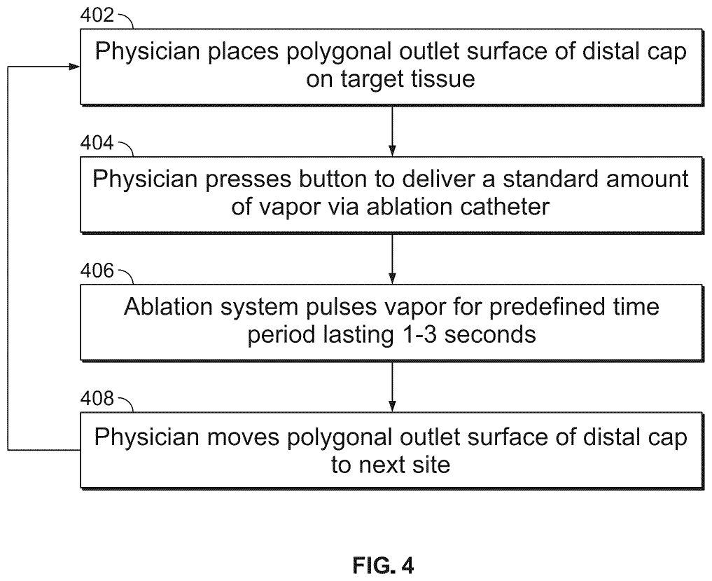

FIG. 4 is a flowchart listing the steps of a method of using an ablation system having a distal cap on an ablation catheter, in accordance with some embodiments of the present specification. At step 402, a physician places the circular or polygonal outlet surface on a target tissue, for example a portion of the patient's GI tract. At step 404, the physician presses a button (such as switch 327 of FIG. 3B or a foot pedal, such as input device 125) that causes the ablation system to pulse a standard amount of vapor. At step 406, the ablation system pulses vapor for a predefined period of time lasting in a range of 0.01-10 seconds. At step 408, the physician moves the circular or polygonal outlet surface to the next site. The physician then continues at step 402 until all focal ablation is completed. In embodiments, the ablation system is configured to output a standard amount of vapor for a predefined time period of 0.01-10 seconds as long as the physician is pressing the button (for example, foot pedal). The vapor pulse continues until the first of 1) the predefined time period (0.01-10 seconds) runs out or 2) the physician stops pressing the button (lifts foot off foot pedal). In another embodiment, where the energy level is input through the user interface, the vapor pulse continues until the first of 1) the predefined amount of energy is delivered (10-500 Joules) or 2) the physician stops pressing the button (lifts foot off foot pedal).

The cap provides for directed, focal ablation and encloses the focal ablation area, optionally (but not preferentially) creating a seal and an enclosed treatment volume for ablation of the tissue. Preferably, the contact of the cap with the tissue area guides, vapor toward the treatment area, such that a portion of the patient's tissue is positioned within an area circumscribed by the attachment, but does not seal the cap over the surface of a patient's tissue, such as the esophagus or duodenum. In embodiments of the present specification, the outer surface 367 outside the circular or polygonal cap has an atraumatic shape. In one embodiment, the exterior periphery 367 of the circular or polygonal cap is rounded or curved so as to avoid sharp surfaces that could potentially damage the patient's GI tract.

In one embodiment, the flow rate of vapor out of the enclosed, or partially enclosed, volume is a predefined percentage of the flow rate of vapor into the enclosed, or partially enclosed volume from the catheter ports, where the predefined percentage is in a range of 1% to 80%, preferably less than 50%, and more preferably less than 30%. The at least one port is positioned at a distal end of the catheter such that it exits into the treatment volume when the attachment is positioned.

The devices and methods of the present specification can be used to cause controlled focal or circumferential ablation of targeted tissue to varying depth in a manner in which complete healing with re-epithelialization can occur. Additionally, the vapor could be used to treat/ablate benign and malignant tissue growths resulting in destruction, necrosis and absorption of the ablated tissue. The dose and manner of treatment can be adjusted based on the type of tissue and the depth of ablation needed. The ablation device can be used not only for the treatment of cardiac arrhythmias, Barrett's esophagus and esophageal dysplasia, flat colon polyps, gastrointestinal bleeding lesions, endometrial ablation, pulmonary ablation, but also for the treatment of any mucosal, submucosal or circumferential lesion, such as inflammatory lesions, tumors, polyps, cysts and vascular lesions. The ablation device can also be used for the treatment of focal or circumferential mucosal or submucosal lesions of any hollow organ or hollow body passage in the body. The hollow organ can be one of gastrointestinal tract, pancreaticobiliary tract, genitourinary tract, respiratory tract, heart, portions of the cardiovascular system, bladder, uterus, or a vascular structure such as blood vessels. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radiopaque or sonolucent material can be incorporated into the body of the catheter for radiological localization. Ferromagnetic materials can be incorporated into the catheter to help with magnetic navigation.

Ablative agents such as saline, steam, heated gas or cryogens, such as, but not limited to, liquid nitrogen can be directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

The ablation systems of the present specification are configured to have reduced start-up times and priming processes. In some embodiments, impedance is measured during start-up to check whether saline is in contact with electrodes. The controller is configured to automate the impedance check and generate an error indicative of a high impedance, signifying that saline or a conductive fluid is not in fluid contact with the electrode(s) or bi-polar electrode or a wire is broken. Rapid changes in impedance level at the electrodes, from high to low, are also detected to be an indication that the saline is in contact with the electrodes. Additionally, in embodiments, the controller is configured to check the power level delivered to the electrodes during the start-up. In embodiments, the controller is configured to check a radiofrequency (RF) power relative to a direct current (DC) power level. Preferably, the controller checks to determine if the RF power matches the DC power in a range of at least 50%, preferably approximately 75%. If not, the controller does not allow the treatment session to continue and indicates an error, such as an electrical short, fluid blockage, or some other error, on the graphical user interface, preferably with instructions on how to resolve the error. Preferably, the controller checks to determine if the motor current is experiencing an increase of more than 10%, or approximately 25%, indicating a stall current torque on motor. If such an increase is determined, the controller does not allow the treatment session to continue and indicates a fluid blockage on the graphical user interface, preferably with instructions on how to resolve the fluid blockage. Embodiments of the present specification also eliminate the need for a pressure sensor. Any fluid blockage or flow issues are detected by measuring resistance of fluid flow at the pump. Relatively higher current needed to push the syringe may indicate a blockage. The detection is therefore performed by the controller, eliminating the need to include a pressure sensor with the catheter.

In embodiments, the controller is configured to automatically flush the catheter before insertion into the patient, during start-up. Automatic flushing by the controller bypasses the need for the user to activate the flushing and manually stop the flushing once the fluid comes out of the catheter. In embodiments, start-up time is decreased by delivering a high power on the order of 2 times to 4 times the normal treatment power level (which could be in a range of 1 W to 150 W-300 W), to kick start the steam and then decreasing the power to a steady state level (which may be approximately 60 W to 80 W or otherwise defined as a power level that does not vary over a time period, such as 5, 10, or more seconds, by more than 10%, and preferably by more than 5%) delivered to the electrodes. The power delivered is also automatically controlled by the controller. In some embodiments, the increase in power level occurs as a result of the start of steam generation process which causes resistance of the fluid (electrical medium) to increase from a range of 1-2 Ohms to approximately a range within 10-20 Ohms. At the resistance of the fluid that is in the range of 1-2 Ohms, the power delivered automatically is 1 to 300 W, which varies spontaneously. Once the fluid starts to boil, the resistance of the fluid increases and the power automatically decreases to a range within 60-80 W.

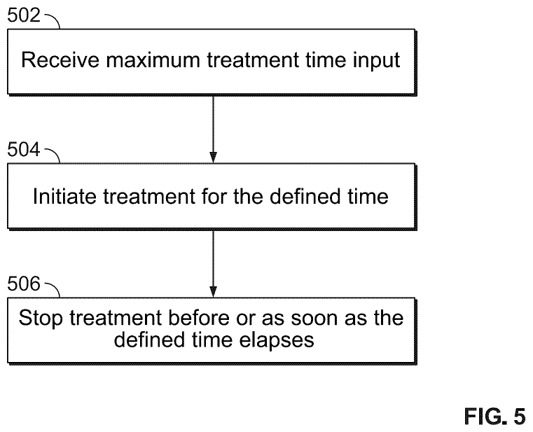

Embodiments of the ablation system of the present specification provide methods and systems for controlled generation of vapor for ablation. Referring again to FIG. 1A, in some embodiments, the ablation system 100 is responsible for generating an electrical current and for applying force to the pump 140 that provides a flow of saline to produce an ablative agent such as saline vapor in the lumen 118 of the catheter 110. The electrical current is passed to electrodes 118a/118b within the lumen 118, with the use of an electrical port 127 that is directly connected to an electrical port 157 on the controller 150. FIG. 5 is a flow chart illustrating an exemplary process of controlling generation of vapor in ablation device 100, in accordance with some embodiments of the present specification. At step 502, a user interface (UI) is available in the form of an input device to the controller 150. A user, such as a clinician, interfaces with the UI to set a maximum treatment time or ablative energy level to be achieved during the vapor ablation process. In embodiments, the treatment time or an energy level to be achieved is set prior to initiating the treatment. The UI may provide a touch screen, buttons, or a combination of both, and a display, to enable the user to input and view the time period being set for the treatment and/or the energy level to be achieved in the treatment, as shown in FIG. 6. At step 504, where the user has input a treatment time, the input value is used by the controller to determine the amount of energy needed to ablate the target tissue. Alternatively, where the user has input an energy level to be achieved during the treatment, the input is used by the controller to determine the amount of time needed to ablate the target tissue. Treatment is initiated when the controller starts supplying power to operate the electrodes 118a and 118b, which in turn generates vapor by heating the ablation fluid supplied by the pump 140. The power supplied to the ablation device 100 varies spontaneously based on a resistance of the fluid, and the rate of flow of the ablation fluid from pump 140 is constant, therefore the amount of time or energy set by the user, which is a function of the tissue being ablated, determines the amount of energy that is delivered at the target site during ablation. An impedance/resistance of the ablation fluid is measured since the flow rate is stable and the salinity (conductivity) of the ablation fluid is consistent. The following equation is used to represent the total energy that is delivered:

Total energy delivered=integral of (Power×Time), where Power is a function of the current and the voltage supplied to the device 100 from a power source.

At step 506, controller 150 stops vapor generation and therefore discontinues the delivery of energy, when the set time period for the treatment completed or the amount of energy to be delivered is achieved. The vapor generation stops as the controller 150 disables the power supply to electrodes 118 in the catheter 110, when the pre-defined time elapses. Optionally, the user may stop the process before the set time period, through manual intervention. In one embodiment an input device provided in the form of an option or a button on the UI, or the foot pedal, is used to intervene and stop the ablation process. Embodiments of the present specification are able to limit the maximum dose of ablation that is delivered by automatic shutting off of the generation of vapor based on a maximum treatment dose. The maximum dose is input into the device 100 as a function of time or delivered energy at step 502. As an additional safety measure, the device 100 enables the user to discontinue delivery of energy at any time during the treatment, even before the maximum dose is reached, by disabling the power supply using the UI or by releasing the pedal. In some embodiments, the controller 150 is programmed to deliver therapeutic ablation treatment repeatedly for a pre-defined duration, where each treatment is for the set time period or energy to be delivered value and is interleaved with a gap of another pre-defined time or energy to be delivered value when the vapor delivery is stopped.

While the catheter preferably does not comprise a sensor to sense the flow rate, temperature, pressure, vapor quality or moisture level, in one embodiment, the catheter may comprise a programmable element or a component with a characteristic that can be measured, such as a resistor. Preferably, the value stored in the programmable element or measured component value, such as a resistor value, can be automatically set by the controller. In one embodiment, the controller is configured to program the value, such as a resistance value, based on a type of treatment or set treatment variables, such as power, voltage, current, fluid flow rate and/or treatment time, as discussed throughout this application.

### Vapor Generation Control Algorithms

In embodiments, the controller is programmed to automate the generation of vapor for priming and for the therapeutic treatment. In one embodiment, a control algorithm emanates from control signals and measurements associated with RF energy delivery to ablation fluid and the resulting transformation of the ablation fluid (saline) into the vapor or steam phase. The controller controls voltage, current and/or power to heat the ablation fluid and generate fluid vapor, and measures the results of these control signals. The measured signals are used to further control or optimize the characteristics of the vapor or steam. Accordingly, the vapor generation process encompasses a series of steps that are performed prior to using the ablation device. A control or an output voltage is set by the user, and the resultant current is measured. The controller then calculates the impedance and resulting power that is delivered to the ablation fluid. Similarly, a control or output current is set, the resulting voltage is measured, and power delivered to the ablation fluid and the resulting impedance are calculated. Further, a control or output power is set, the voltage or current levels are adjusted to achieve the desired power to be delivered to the ablation fluid, and the resulting impedance is calculated. Still further, a control or output voltage is set, the resultant current is measured, the power delivered to the fluid to produce vapor and the resulting impedance are calculated. In embodiments, the control voltage and/or the control current can be adjusted to modify the power delivered to the fluid as well as the amount or quality of steam that is generated.

As mentioned here, the controller is enabled to measure and control steam generation using one or a combination of the stated steps. In the cases where the controller a) sets the control voltage and measures the current, the impedance is calculated, and b) sets the control current and measures the voltage, the impedance is calculated, a change in impedance is calculated when vapor generation is initiated resulting in an associated change in the impedance. The change in impedance may reflect as a sharp increase in impedance, for example in the form of a ‘step change’. The amount of the change in impedance may depend on the intrinsic impedance of the fluid. For example, for saline the intrinsic impedance at the RF output frequency of 460 kHz for one steam chamber geometry is on the order of 2 ohms. When generating vapor, the impedance at 460 kHz is highly variable and on the order of, for example, 10-60 ohms or larger, depending on the vapor quality and the vapor power.

A start of vapor or steam generation may be detected based on one or more characteristics derived from measurement of impedance. In one embodiment, step change in impedance from approximately 2 ohms to a minimum average value of approximately 15 ohms may indicate vapor generation. In another embodiment, change in the characteristics of the impedance calculations from a semi constant or slowly changing value on the order of 1-3 ohms to a highly variable value ranging from approximately 10 ohms to 60 ohms, or more, indicates vapor generation. The change in impedance is caused by the conversion of a conductive fluid to vapor, or a conductive vapor to a higher ‘quality’ vapor, as the random process of boiling occurs with the resulting vapor that is in contact with the electrode surfaces being less conductive than the fluid that was in contact with the electrode surfaces. Since boiling is random, the resulting impedance changes randomly from a low value to high values and values between the low and high ranges.

In the case where the controller:
a. sets the control voltage and measures the current,
b. sets the control current and measures the voltage
the RF output power is calculated. A change in output power is instantaneously achieved and can be detected when the vapor generation begins. The change in output power is reflected as a sharp decrease, or a ‘step change’ decrease in output power. The amount of the change may depend on the intrinsic impedance of the ablation fluid. For example, for saline the intrinsic impedance at the RF output frequency of 460 kHz is on the order of 2 ohms for a certain model of the steam chamber geometry. When delivering the vapor the impedance at 460 kHz is variable and on the order of, for example, 1-60 ohms or larger, depending on the vapor quality and the vapor power.

The controller may detect the start of actual vapor or steam generation based on one or more characteristics derived from measurement of power. In one embodiment, a decrease or step change in output power from approximately 100-200 watts to a minimum average value of approximately 60-80 Watts indicates vapor generation. In another embodiment, a change in the characteristics of the power calculations from a dynamically changing value on the order of 1-200 Watts to a variable value ranging from approximately 10 Watts to 60 Watts, is an indication of vapor generation. While the changes in the calculated power depend on the voltage setting but the form of the changes are similar. For example, if the voltage is on the order of 30 volts, the average power is calculated on the order of 60 Watts. If the voltage is on the order of 15 volts, the average power is calculated on the order of 20 Watts. Accordingly, in one embodiment, the controller is configured to detect the actual start of vapor generation, as independent and separate from the initiation of fluid flow to a heating chamber, by monitoring a change in output power, such as a decrease in a first output power from a range of 1 to 200 watts to a second output power in a range of 60 watts or less.

In the case where the controller sets the average power and adjusts the current and/or the voltage to achieve the set average power, a change in output voltage and/or current is detected when the vapor generation begins. The change in output voltage and/or current or resistance is reflected as a sharp increase, or a ‘step change’ increase in voltage, or sharp decrease or a ‘step change’ decrease in current. The amount of the change may depend on the intrinsic impedance of the ablation fluid. For example, for saline the intrinsic impedance at the RF output frequency of 460 kHz is on the order of 2 ohms for a certain model of the steam chamber geometry. When delivering the vapor the impedance at 460 kHz is variable and on the order of, for example, 1-60 ohms or larger, depending on the vapor quality and the vapor power. Accordingly, in one embodiment, the controller is configured to detect the actual start of vapor generation, as independent and separate from the initiation of fluid flow to a heating chamber, by monitoring a change in output voltage or current, such as an increase in output voltage or decrease in current.

The controller may detect the start of actual vapor or steam generation based on one or more characteristics derived from measurement of power. In one case, if there is a decrease or step change in average output power from approximately 100-200 watts to a minimum average value of approximately 40-50 watts, change in the characteristics of the power calculations from a semi constant or minimally changing value on the order of 100-200 watts to a highly variable value ranging from approximately 10 watts to 60 watts, is an indication of vapor generation. While the changes in the calculated power depend on the voltage setting, the directionality of the changes are similar. For example, if the voltage is on the order of 30 volts, the average power is calculated on the order of 50 Watts. If the voltage is on the order of 15 volts, the average power is calculated on the order of 20 Watts.

In another case, if there is an increase or step change in resistance from approximately 2-3 ohms to a minimum average value of approximately 8-20 Ohms, change in the characteristics of the resistance values from a semi constant or minimally changing value on the order of 2-3 Ohms to a highly variable value ranging from approximately 8 Ohms to 20 Ohms is an indication of vapor generation. In yet another embodiment, if there is an increase or step change in voltage from approximately 7 volts to a minimum average value of approximately 30 volts, change in the characteristics of the output voltage from a semi constant or minimally changing value on the order of 7 volts to a highly variable value ranging from approximately 30 volts to 34 volts, is an indication of vapor generation. Accordingly, in one embodiment, the controller is configured to detect the actual start of vapor generation, as independent and separate from the initiation of fluid flow to a heating chamber, by monitoring a change in the variability of resistance, a change in the variability of current, a change in the variability of voltage, or a change in the variability of power, such as an increase in variability.

For each of the above-stated modes of controls involving voltage, current, resistance and power, the voltage source control can be replaced with a current source control and associated measurements of voltage, to achieve the same control responses. Alternatively, for each of the control modes the current source control can be replaced with a voltage source control and associated measurements of current, to achieve the same control responses. Additionally, voltage or current control can be replaced with power control and the associated changes in the control voltage or control current can be achieved.

In embodiments of the present specification, the RF voltage, current and/or power delivery is interrupted, and therefore not constant, during the treatment time, which has the effect of decreasing the energy delivered and may alter or reduce the resultant temperature of the fluid or vapor, and/or reduce the rate of fluid or vapor temperature increase during the treatment time. The interruption in RF energy delivery may occur in a periodic or non-periodic manner to result in the desired energy delivery profile and/or rate of temperature increase. Additionally, the fluid flow rate may be modified in a periodic or a non-periodic manner to adjust the energy delivery rate and/or rate of temperature rise.

Embodiments of the present specification enable the controller to be programmed to control a pulsed delivery of ablative energy in the form of vapor, and the temperature response, during a therapeutic treatment. For this purpose, the user may set a treatment time, which would then automatically result in the setting of voltage, current, and/or power at a level that is desired for ablating a target tissue in accordance with the set time. In one embodiment, for a specific configuration of the steam chamber of an ablation device, the control voltage, current or power is automatically set for a portion of the inputted time period, where that portion is approximately 250 milliseconds (ms). The controller may then automatically stop the RF delivery for a period of approximately 250 ms. The duty cycle of 250 ms of enabling RF delivery and 250 ms of disabling RF delivery is repeated. In this way, a pulsed delivery of RF energy is provided to the ablation device that represents a periodic and symmetric 50:50 duty cycle. In another embodiment, the control voltage, current or power automatically set by the inputted time is delivered for a period of approximately 900 ms, followed by a gap of approximately 300 ms when the RF delivery is stopped, followed again by RF delivery of 900 ms. In this embodiment, repeating the duty cycle of 75:25 also delivers pulsed ablative energy. It should be appreciated that the duty cycle of on: off may be divided into 5:95 to 95:5 as a proportion of the inputted treatment time. As such, the controller is configured to automatically translate an inputted treatment time into a on: off duty cycle in the aforementioned ranges.

In further embodiments, the duration of enabling and disabling the RF delivery may be varied within a treatment duration. In one embodiment of a pulsed delivery cycle, the control voltage, current, or power is delivered for 100 ms, stopped for 100 ms, delivered again for 100 ms, then stopped for 300 ms. Repeating this pulsed delivery pattern represents a periodic and asymmetric duty cycle of 50:50:25:75. Therefore, in embodiments, the duty cycle may be adjusted to result in a multitude of energy delivery and/or rate of fluid and/or vapor temperature rise results.

While not preferred, the output from the ablation device is measured during the treatment to provide additional controls, in accordance with some embodiments. In some embodiments, a flow rate of the pump, such as a syringe pump, is adjusted during the treatment. Adjusting the flow rate may optimize vapor quality so as to increase average impedance. The flow rate is adjusted to increase or decrease based on the required quality of vapor. An increased flow rate decreases the vapor quality whereas a decreased flow rate increases the vapor quality, provided a fixed voltage or current is being used. In one scenario, the flow rate is decreased or even ceased so as to minimize fluid delivery after the ablative vapor is delivered to the target tissue, such as in a pulsed delivery of treatment. In other embodiments, the controller can receive output signals, such as temperature and impedance during vapor generation, and use them to adjust or control the fluid flow rate from the pump. Output measurements from the ablation device can also be used to monitor consistency of vapor generation, and fluid flow rate control signals can be adjusted to compensate for output variations.

In one embodiment, the fluid flow rate is adjusted by the controller to a first rate (R1) before detecting the generation of vapor; and to a second rate (R2) after detecting the generation of vapor. Such modification of the rate of flow from syringe pump can minimize delivery of fluid or low quality vapor to the treatment site. In one example, the fluid flow rate (R1) before detection of vapor production may be approximately 0.01 milliliter per minute (ml/min) to 1.0 ml/min and the fluid flow rate (R2) after detection of vapor may be approximately 2.0 ml/min to 2.2 ml/min. The flow rate may vary for different geometries of steam chambers. Multiple combinations are possible depending on the desired vapor power output, quality and amount of liquid delivered to the treatment site.

### Alternative Embodiment

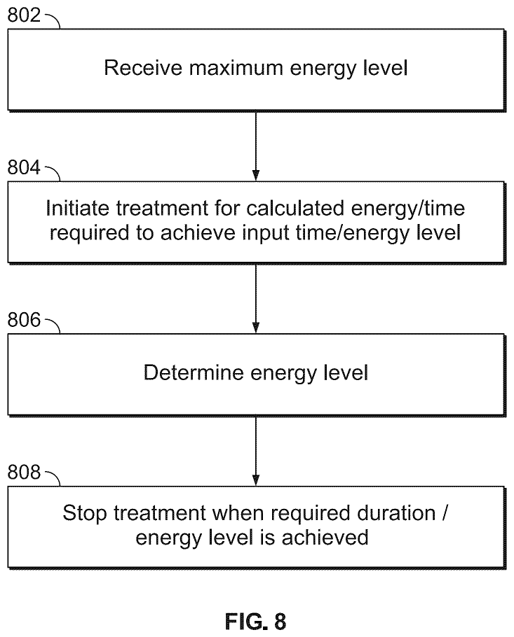

Embodiments of the ablation system of the present specification provide methods and systems for controlled generation of vapor for ablation. Referring again to FIG. 1A, in some embodiments, the ablation system 100 is responsible for generating an electrical current and for applying force to the pump 140 that provides a flow of saline into the lumen 118 of the catheter 110 and thus produce an ablative agent such as steam vapor. The electrical current is passed to electrodes 118a/118b within the lumen 118, with the use of an electrical port 127 that is directly connected to an electrical port 157 on the controller 150. FIG. 8 is a flow chart illustrating an exemplary process of controlling generation of vapor in ablation device 100, in accordance with some embodiments of the present specification. At step 802, a user interface (UI) is available in the form of an input device to the controller 150. A user, such as a clinician, interfaces with the UI to set a maximum energy limit that should be delivered by ablation. In one example, the energy limit is set to 300 Joules. The controller calculates the energy as an integral of the product of power and time. Power is calculated as per the following equation:

Total energy delivered=integral of (Power×Time), where Power is a function of the current and the voltage supplied to the device 100 from a power source.

The time is adjusted by the controller to achieve the desired energy limit based on the fixed voltage and the measured current. The power output levels of different catheters vary, therefore the time is adjusted to achieve a consistent energy level. When the power is known, the controller delivers power for the specified time, and then stops. In one embodiment, where an average power output of a catheter is 50 W, the controller is configured to deliver RF for approximately 6 seconds when the total energy limit is set to 300 J. The vapor delivery is terminated when the controller determines the energy limit has been reach.

Accordingly, the present system can be easily customized to use different types of catheters by a) applying a known, fixed voltage, b) measuring the resulting current prior to conducting an ablation procedure, c) determining the power as a function of the fixed voltage and measured current, d) determining a preferred time for an ablation procedure based on a desired, target amount of power and the calculated power, and c) performing an ablation procedure using the preferred time, e) determining a preferred delivered total energy for an ablation procedure based on the target amount of power and the calculated power, and c) performing an ablation procedure using the preferred energy. In another embodiment, the present application discloses a method of sequentially performing two or more ablation procedures using the same generator but with different catheters. In the first ablation procedure, a first catheter is attached to a generator, as described herein. The generator a) applies a known, fixed voltage, b) measures the resulting current through the first catheter prior to conducting the first ablation procedure, c) determines the power as a function of the fixed voltage and measured current, d) determines a first preferred time or preferred delivered energy for the first ablation procedure based on a desired termination end point or target amount of power specific to that first ablation procedure and the calculated power, and c) enables the first ablation procedure to be used with the first catheter using the preferred time or delivered energy. In a second, subsequent ablation procedure, a second catheter is attached to the same generator, as described herein, wherein the second catheter is different from the first catheter. The generator a) applies a known, fixed voltage, b) measures the resulting current through the second catheter prior to conducting the second ablation procedure, c) determines the power as a function of the fixed voltage and measured current, d) determines a second preferred time or delivered energy for the second ablation procedure based on a desired, target amount of power specific to that second ablation procedure and the calculated power, and c) enables the second ablation procedure to be used with the second catheter using the preferred time or delivered energy. In one embodiment, the target power for the first ablation procedure is different from the target power for the second ablation procedure and the preferred times or delivered energy for both procedures are different.

Regarding a method of operation, at step 804, treatment is initiated when the controller starts supplying power to operate the electrodes 118, which, in turn, generate vapor by heating the ablation fluid supplied by the pump 140. A portion of the duty cycle when the RF energy starts to be delivered with the supply of power, is used to improve the delivery of vapor. The vapor delivery is improved by increasing or decreasing the flow rate of the pump. Gradually increasing the flow rate in the first portion of the time for which the vapor is delivered, ensures a consistent build-up of vapor resulting in a faster wetting of the catheter electrode so that there is no air or contaminants during the vapor-building process. Conventionally, vapor production is started by starting first the delivery of RF energy and then starting fluid flow or by initiating fluid flow and RF energy simultaneously. However, the conventional methods of vapor production sometimes result in vapor sputtering at the start of the procedure. Embodiments of the present specification address this problem by improving the start of vapor production so that vapor is generated within 0.6 to 0.8 seconds of starting the delivery of RF energy, during which time the syringe pump is pushed faster to create more instantaneous pressure more immediately, thereby increasing the flow rate during the first half (approximately) of a second. In an embodiment, in the first 20% of procedure time, the fluid flow rate is 30% higher (on the order of 2 times to 3 times the flow rate during the remaining procedure time) relative to the remaining 80% of the procedure time.

At step 806, in embodiments, the controller is enabled to be programmed to start detection and measurement of the therapeutic energy delivered by vapor using the calculation of power supplied and time taken to deliver vapor, as described above. After a first time period (when the pump pressure is greater to create a higher instantaneous pressure), a drop in pump flow rate and thus, pressure, is experienced during a subsequent second time period when vapor production is initiated. After vapor production is initiated, the system experiences an increase in pump pressure during a subsequent third time period. Throughout the first, second, and third time periods, the system is constantly measuring current and fluid flow or impedance and, based on a fixed voltage, is therefore able to measure power in real-time throughout each of the first, second, and third time periods. Accordingly, during the first time period, the system will experience a first power in a range of 1 W to 200 W. During the second time period, the system will experience a second power in a range of 40 W to 60 W, and during the third time period, the system will experience a third power in a range of 40 W to 60 W. Alternatively, it should be appreciated that the second power range will be approximately 60% less than the first power range while the third power range will be approximately equal to the second power range.

The system controller can be configured to identify a change in power consumption. When the system controller identifies a change in average power consumption from 1-200 W to 40-60 W, the system controller may conclude that vapor production has initiated. Once the system controller determines vapor production has started, based on monitoring power consumption, the controller can initiate a timer to count and track the preferred time or delivered energy, which is calculated as discussed above in order to deliver the target energy level, such as 300 J, only after vapor production has initiated. Therefore, embodiments of the present specification have the ability to detect the point in time when vapor is being produced without using a separate sensor, based on characteristics of the power delivery parameters, such as power, current, voltage, and impedance, and use that vapor production initiation point to start the treatment session timer. As a result, a consistent level of energy may be provided, following an initial spike in power level, for the calculated time period without requiring a vapor detection sensor in the catheter.

Additionally, an amount of power generation can be controlled by controlling the salinity of the ablation fluid and its resistivity. Salinity of the fluid may be adjusted to achieve different outputs, for example, in one embodiment, higher salinity will require an increase in current to produce higher quality vapor. Moreover, different end points, can be achieved with different levels of salinity. In one example, diluting 0.9% normal saline with water to approximately 0.7% can increase the average power at the same voltage by approximately 30%.

At step 808, controller 150 stops vapor generation and therefore discontinues the delivery of energy, when the set time period or desired energy delivery is completed. Conventionally, at the end of an ablation treatment, the flow of fluid is stopped immediately. As a result of stopping the flow of fluid, the vapor that was being generated and had already been generated within the catheter, condenses, and air or other contaminants can be aspirated into the electrode region. The electrode in contact with the contaminants with air and fluid is herein referred to as a ‘dry/dryer/dried electrode’, all used synonymously. A dried electrode may result in air or other contamination to exist in the treatment region. Contamination is avoided by continuing to flush the catheter lumen with fluid such as water or saline after the power delivery ceases, or significantly decreases. Embodiments of the present specification overcome the limitations of the conventional methods of ending the treatment by ramping down the voltage while simultaneously increasing the rate of flow of fluids, instead of immediately turning off the fluid pump and/or the power supply.

Accordingly, after the calculated time period or delivered energy, as described above, ends, the treatment is deemed to be terminated and the power supply is shut down over a period of time while the flow of fluid is temporarily increased. In one example, the fluid flow rate is increased by a factor of approximately 1.25 times to 3 times relative to the fluid flow rate during vapor generation and the power is reduced and ultimately shut off within a few hundreds of milli seconds to a few seconds, thus preventing the vacuum force that can lead to aspiration of air and/or other contaminants. The vacuum can result from a specific volume of steam vapor collapsing to water/saline by a factor of 1500:1 when vapor production stops. Embodiments of the present specification increase fluid flow which ‘extinguishes’ the vapor with fluid, rather than only shutting off power and allowing the steam to instantaneously collapse to a fluid. A controlled combination of voltage and flow rate, in accordance with the embodiments of the present specification, reduces the vacuum effect. As a result of ramping down the voltage, and therefore power supply, and controlling the flow of ablation fluids, aspiration into the catheter device is minimized, after delivery of the therapeutic vapor. Additionally, the stated methods of ending the treatment after flush and drip functions within the catheter, keep the vapor generation elements clean and ready to produce vapor for a subsequent treatment. Moreover, embodiments of the present specification prevent the back flow of contaminants, such as for example contaminants like air, fluids, or any other types of contaminants, into the catheter and/or vapor generation area.

Embodiments of the present specification are able to limit the maximum dose of ablation that is delivered by automatic shutting off of the generation of vapor based on a maximum treatment dose. As an additional safety measure, the device 100 enables the user to discontinue delivery of energy at any time during the treatment, even before the maximum dose is reached, by disabling the power supply using the UI or by releasing a pedal. In some embodiments, the controlled 150 is programmed to deliver a defined single treatment, the catheter is relocated, and a new treatment is delivered to another area. After a predetermined time period, which, in some embodiments, is in a range of 1 second to 10 minutes, and more preferably 1 minute to 2 minutes, the catheter is returned to the previously treated area and the area is retreated. This results in a more complete and uniform ablation area, without increasing the risks associated with deeper thermal injury. In other embodiments, the controller 150 is programmed to deliver therapeutic ablation treatment repeatedly for a pre-defined duration, where each treatment is for the set time period and is interleaved with a gap of another pre-defined time when the vapor delivery is stopped. Therefore, in embodiments, the total treatment time, as described above, is automatically divided into a plurality of vapor pulses, separated by a predefined time period, such that the sum of the time of the individual pulses equals the total treatment time calculated above. Vapor generation is thus achieved discretely in a pulsed, and nearly ‘square wave’ form without an active or passive valve mechanism.

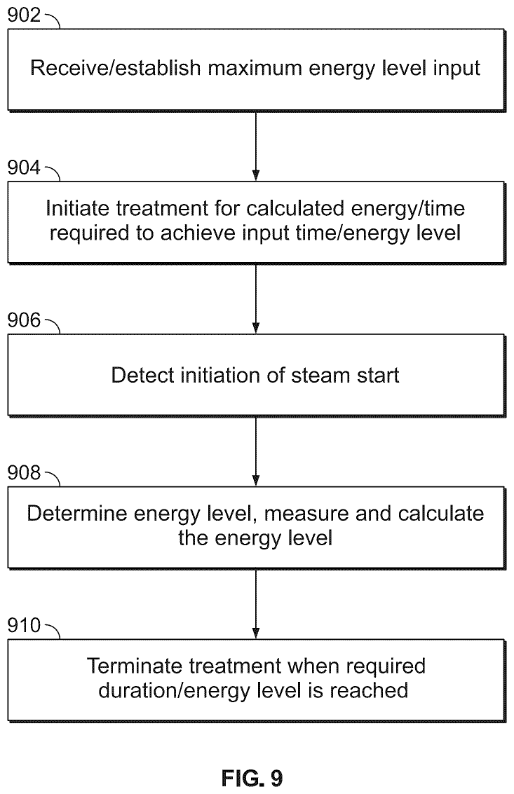

FIG. 9 is a flow chart illustrating another exemplary process of controlling generation of vapor in an ablation device wherein the energy level is inputted to the controller, in accordance with some embodiments of the present specification. At step 902, a maximum energy level input is received or established by an ablation device. In embodiments, the maximum energy level is entered by a user into a controller of an ablation system comprising the ablation device. In some embodiments, the maximum energy level is preset into a controller of an ablation system comprising the ablation device. In some embodiments, the preset maximum energy level is based on a catheter of the ablation device. In embodiments, the maximum energy level is related to a duration to apply the maximum energy and time is used as an end point for treatment. At step 904, treatment for a calculated energy/time required to achieve the input time/energy level is initiated. At step 906, the initiation of steam start is detected. In embodiments, the initiation of steam defines the start of the energy count to the maximum energy level. At step 908, the controller is configured to measure and calculate the energy being applied to determine the energy level. At step 910, the controller is configured to terminate treatment when the required duration/energy level is reached.

Embodiments of the present specification combine the use of a pair of bipolar electrodes to directly drive energy into the fluid between the two electrodes thereby generating vapor, and using an energy level to be achieved as a basis for controlling the ablation process. Several benefits are obtained from said combination: Firstly, since the fluid directly receives power and there is no intermediate heating or cooling step, the ablation fluid can be instantly vaporized and then the vapor generated can be instantly turned off. Secondly, catheters of a same type may have different loads and vapor generation characteristics; by focusing on total energy delivery, the ablation performance can be standardized. Stopping the treatment when the input energy level is achieved, provides consistent/reliable ablation temperatures. Thirdly, embodiments of the present specification do not need temperature or steam quality sensors. The precise relationship between power generated and power into the fluid provides for a precise way of determining the temperature and steam quality without requiring sensors. Further, a certain rate of fluid flow is a key variable to determining effects of the procedure for a certain volume of tissue.

Configurations for the various catheters of the ablation systems of the embodiments of the present specification may be different based on the tissue or organ systems being treated. Distribution and depth of ablation provided by the systems and methods of the present specification are dependent on the duration of exposure to steam, the ablation size, the temperature and/or quality of the steam, the contact time with the steam, and the tissue type.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of performing an ablation procedure, comprising:
inputting at least one of a treatment time or an energy level into a controller, wherein the controller is in fluid communication with a pump and in electrical communication with a catheter and wherein the catheter has an elongate shaft, a proximal end, and a distal end, at least one lumen within the elongate shaft and at least one bipolar electrode positioned within the lumen;
causing the controller to generate an electrical current and direct the electrical current to the at least one bipolar electrode such that the electrical current passes through the fluid positioned proximate the at least one bipolar electrode and causes the fluid to be transformed to a heated vapor, wherein the fluid is not fully transformed to heated vapor via resistive heating of the at least one bipolar electrode; and
controlling a flow rate of the fluid and at least one of a level of power, voltage, current, and a time of treatment, based on data indicative of at least one of the time or the energy level to be achieved within the treatment session.

2. The method of claim 1, further comprising not using temperature sensors or quality of steam sensors in the catheter.

3. The method of claim 1, further comprising, prior to initiating treatment of a patient, using the controller to apply a known voltage and measuring a resulting current.

4. The method of claim 3, further comprising determining power as a function of the known voltage and the measured resulting current.

5. The method of claim 4, further comprising determining a preferred time for the ablation procedure based on a target amount of power and the determined power and performing the ablation procedure using the preferred time.

6. The method of claim 4, further comprising determining a preferred energy for the ablation procedure based on a target amount of power and the determined power and performing the ablation procedure using the preferred energy.

7. The method of claim 6, wherein the controller is configured to terminate electrical current generation when a total energy level associated with all of the heated vapor has reached the preferred energy.

8. The method of claim 1, wherein the controller is configured to cause a first flow rate of the fluid before a generation of the heated vapor starts and a second flow rate of the fluid after the generation of the heated vapor starts, wherein the first flow rate is greater than the second flow rate.

9. The method of claim 8, wherein the controller is configured to cause a third flow rate of the fluid after the preferred energy has been achieved, wherein the third flow rate is greater than the second flow rate.

10. The method of claim 1, further comprising directing heated vapor through a cap in fluid communication with the distal end of the catheter, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater.

11. The method of claim 10, wherein the cap comprises rounded exterior edges and is removably attachable to the distal end of the catheter.

12. The method of claim 10, wherein the sole opening is polygonal in shape and wherein the polygonal shape comprises one of a square, a rectangle, a pentagon, or a hexagon.

13. The method of claim 10, wherein the cap is angled relative to the longitudinal axis of the catheter in a range of 5 degrees to 90 degrees.

14. The method of claim 1, wherein the catheter further comprises at least one first positioning element comprising a first disk spaced from a second positioning element comprising a second disk wherein the at least one bipolar electrode is positioned within the lumen between the first circular disk and the second circular disk, such that the heated vapor is generated in the space between the first circular disk and the second circular disk.

15. The method of claim 1, further comprising detecting a start of heated vapor generation by monitoring a change in at least one of output power, output voltage, resistance, and output current and not based on an initiation of a flow of fluid.

16. The method of claim 1, wherein the fluid comprises sodium chloride in a range of 0.1% to 50% in water.

17. The method of claim 1, wherein delivering power to the at least one bipolar electrode in a range of 1 watts to 500 watts, and wherein the power is based on at least one of a size of the lumen and a surface area of the at least one bipolar electrode.

18. The method of claim 1, wherein the catheter does not comprise sensors configured to detect data indicative of vapor quality, temperature, moisture level, or pressure of the heated vapor.

19. The method of claim 1, further comprising causing the heated vapor to be generated for a first time period, ceasing a delivery of the fluid for a second time period, and repeating the generation of heated vapor and ceasing of fluid delivery for a plurality of cycles.

20. The method of claim 1, wherein the catheter comprises a programmable element and wherein the controller is configured to program the programmable element based on at least one of a treatment type, the power level, the voltage level, the current level, the fluid flow rate, the treatment time, or the energy level to be achieved.

21. The method of claim 20, wherein the programmable element is a resistor.
